(19) United States
(12) Patent Application Publication
(10) Pub. No.: US 2017/0145472 A1
(43) Pub. Date: May 25, 2017

(54) METHOD OF DETECTING HELICASE ACTIVITY

(71) Applicants: University of Macau, Macau (CN); Hong Kong Baptist University, Kowloon (HK)

(72) Inventors: Dik-Lung Ma, Kowloon (HK); Chung-Hang Leung, Macau (CN); Hai-Jing Zhong, Macau (CN); Bingyong He, Kowloon (HK)

(73) Assignees: Hong Kong Baptist University; University of Macau

(21) Appl. No.: 14/950,538

(22) Filed: Nov. 24, 2015

(51) Int. Cl. C12Q 1/68; G01N 21/64

(52) U.S. Cl. CPC C12Q 1/68; G01N 21/6428; G01N 2333/914; C12Y 306/04012; G01N 2021/6432; G01N 2021/6439

(57) ABSTRACT

The present invention provides a method of detecting helicase activity in a test solution, providing a substrate solution containing a double-stranded DNA, wherein said double-stranded DNA is a substrate for a helicase to be detected; (b) introducing the substrate solution to the test solution to form a reaction solution; (c) applying a luminescent probe into the reaction solution to form a mixture; and (d) measuring a luminescent response of the luminescent probe in the mixture, wherein the luminescent response corresponds to the helicase activity in the test solution. The present invention also refers to a method of screening a potent luminescent probe for detecting helicase activity, and a series of luminescent Ir (III) complexes. Said luminescent Ir (III) complex comprises an iridium-containing dimer and a nitrogen-containing ligand

### TECHNICAL FIELD

The present invention relates to a method of detecting helicase activity, in particular but not exclusively, relates to a method of detecting helicase activity by using a luminescent probe. The present invention also refers to a method of screening a luminescent probe, and a series of metal complexes.

### BACKGROUND

Helicases unwind double-stranded DNA (dsDNA), double-stranded RNA (dsRNA) or displace nucleic acid-binding proteins by using energy from ATP hydrolysis. Helicase is an essential enzyme in cells for the reading, replication, and repair of genomes. However, helicases are also implicated in a number of viral diseases due to their critical role in facilitating viral replication and proliferation. Viral helicase inhibitors have been developed for the treatment of hepatitis C and herpes simplex viral infections. Due to its biological and medical importance, the development of efficient assays for monitoring the nucleic acid unwinding activity of helicase is of great interest.

Conventional techniques for the detection of helicase activity typically involve radioactive labeling in conjunction with gel electrophoresis. However, this method is discontinuous, time-consuming, inefficient, and necessitates the use of stringent safety procedures to control radiographic exposure, thus limiting the scope of their application.

Over the past several years, oligonucleotides have been considered as attractive signal transducing units for the detection of biologically and environmentally important analytes. Oligonucleotides offer salient advantages in biosensing applications, such as their relatively small size, low cost, facile synthesis and modification, good thermal stability, and reusability. In particular, the G-quadruplex motif, which is a non-canonical DNA secondary structure composed of planar stacks of four guanines stabilized by Hoogsteen hydrogen bonding, has attracted particular interest in sensing applications. The extensive structural polymorphism of G-quadruplexes has rendered them as versatile signal-transducing elements for the development of DNA-based probes.

Min and co-workers in H. Jang, Y.-K. Kim, H.-M. Kwon, W.-S. Yeo, D.-E. Kim and D.-H. Min, Angew. Chem. Int. Ed, 2010, 49, 5703-5707; and in H. Jang, S.-R. Ryoo, Y.-K. Kim, S. Yoon, H. Kim, S. W. Han, B.-S. Choi, D.-E. Kim and D.-H. Min, Angew. Chem. Int. Ed, 2013, 52, 2340-2344, have reported a fluorescent assay for hepatitis C virus (HCV) NS3 helicase activity and severe acute respiratory syndrome coronavirus (SARS-CoV, SCV) helicase nsP13 activity by utilizing graphene and a fluorescently-labelled dsDNA substrate. Ali and co-workers in S. Siddiqui, I. Khan, S. Zarina and S. Ali, Enzyme Microb. Technol, 2013, 52, 196-198 have utilized SYBR Green dye, which is fluorescent in the presence of dsDNA but not ssDNA, for the detection of helicase activity. A similar principle was utilized by Kowalczykowski and co-workers to construct a switch-off platform for helicase activity using ethidium bromide, ethidium homodimer, bis-benzimide (DAPI), Hoechst 33258 or thiazole orange. The groups of Frick and Boguszewska-Chachulska in C. A. Belon and D. N. Frick, BioTechniques, 2008, 45, 433-440; and A. M. Boguszewska-Chachulska, M. Krawczyk, A. Stankiewicz, A. Gozdek, A.-L. Haenni and L. Strokovskaya, FEBS Lett., 2004, 567, 253-258 have reported an approach for monitoring helicase activity using molecular beacons. Recently, Balci and coworkers in K. S. Lee, H. Balci, H. Jia, T. M. Lohman and T. Ha, Nat. Commun., 2013, 4, 1878-1887; J. B. Budhathoki, S. Ray, V. Urban, P. Janscak, J. G. Yodh and H. Balci, Nucleic Acids Res., 2014, 42, 11528-11545; and H. Balci, S. Arslan, S. Myong, Timothy M. Lohman and T. Ha, Biophys. J., 2011, 101, 976-984 utilized single-molecule Forster resonance energy transfer (FRET) imaging to monitor helicase activity. Although these reports demonstrate that DNA oligonucleotides may be integrated as functional and structural elements for the construction of luminescent platforms for the detection of helicase, there still remains a need for developing a label-free and sensitive method for determining helicase activity in a more cost-effective and highly efficient manner.

### SUMMARY OF THE INVENTION

In accordance with a first aspect of the present invention, there is provided a method of detecting helicase activity in a test solution, comprising the steps of: (a) providing a substrate solution containing a double-stranded DNA, wherein said double-stranded DNA is a substrate for a helicase to be detected; (b) introducing the substrate solution to the test solution to form a reaction solution; (c) applying a luminescent probe into the reaction solution to form a mixture; and (d) measuring a luminescent response of the luminescent probe in the mixture, wherein the luminescent response corresponds to the helicase activity in the test solution.

Preferably, in step (b), the double-stranded DNA is unwound to provide a single-stranded DNA and a G-quadruplex-forming DNA in the presence of the helicase, in which the G-quadruplex-forming DNA can fold to form a G-quadruplex DNA.

More preferably, the luminescent probe interacts with the G-quadruplex DNA to generate the luminescent response.

It is preferable that the G-quadruplex DNA comprises a sequence of SEQ ID NO: 1.

Advantageously, the luminescent probe is a luminescent iridium (III) complex, the luminescent iridium (III) complex comprises: an iridium-containing dimer; and a nitrogen-containing ligand having at least two nitrogen atoms, wherein the nitrogen-containing ligand is a derivative of pyridine.

More advantageously, the iridium-containing dimer ligand has at least two benzene rings; and the nitrogen-containing ligand is selected from the group consisting of 2,9-diphenyl-1,10-phenanthroline, 2,9-dimethyl-4,7-diphenyl-1,10-phenanthroline, 5,6-dimethyl-1,10-phenanthroline, S-chloro-1,10-phenanthroline, 4,7-dichloro-1,10-phenanthroline, 5,5′-dimethyl-2,2′-bipyridine, 4,7-diphenyl-1,10-phenanthroline, 4,4′-diphenyl-2,2′-bipyridine, pyrazino[2,3-f][1,10]phenanthroline and a derivative thereof.

Preferably, the test solution comprises cells.

It is further preferable that the helicase is hepatitis C virus helicase.

In accordance with a second aspect of the present invention, there is provided a luminescent iridium (III) (Ir(III)) complex comprising, an iridium-containing dimer; and a nitrogen-containing ligand having at least two nitrogen atoms, wherein the nitrogen-containing ligand is a derivative of pyridine.

Preferably, the nitrogen-containing ligand is selected from the group consisting of 2,9-diphenyl-1,10-phenanthroline, 2,9-dimethyl-4,7-diphenyl-1,10-phenanthroline, 5,6-dimethyl-1,10-phenanthroline, 5-chloro-1,10-phenanthroline, 4,7-dichloro-1,10-phenanthroline, 5,5′-dimethyl-2,2′-bipyridine, 4,7-diphenyl-1,10-phenanthroline, 4,4′-diphenyl-2,2′-bipyridine, pyrazino[2,3-f][1,10] phenanthroline and a derivative thereof.

It is preferable that the iridium-containing dimer comprises at least two benzene rings. More preferably, the iridium-containing dimer comprises a chemical structure selected from the group consisting of the following structures:

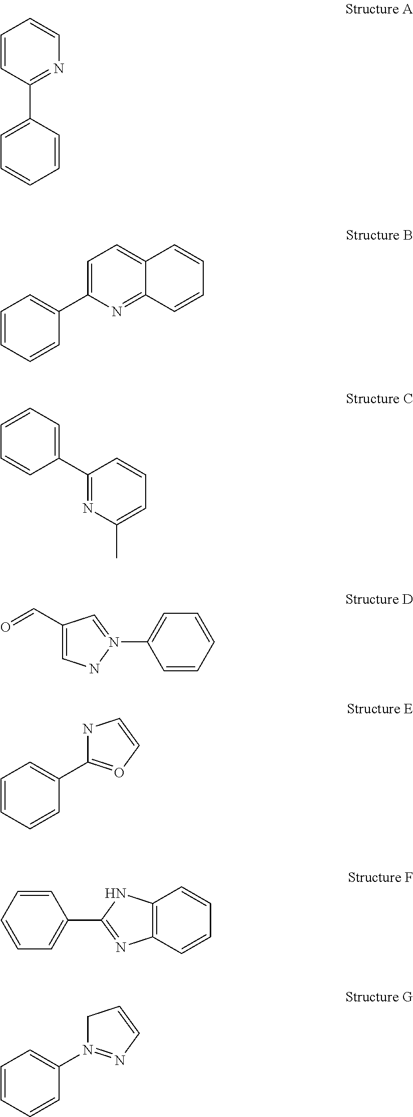

Advantageously, the iridium (III) complex comprises one of the following structures:

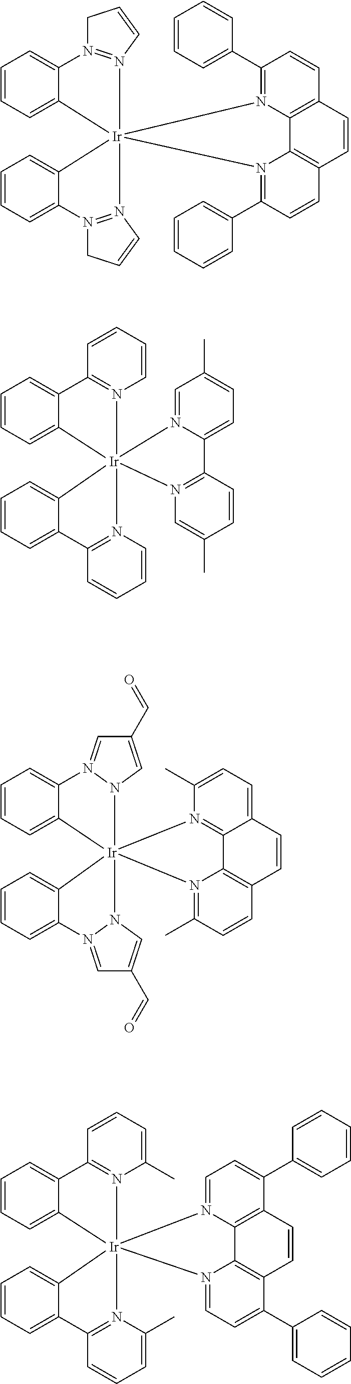

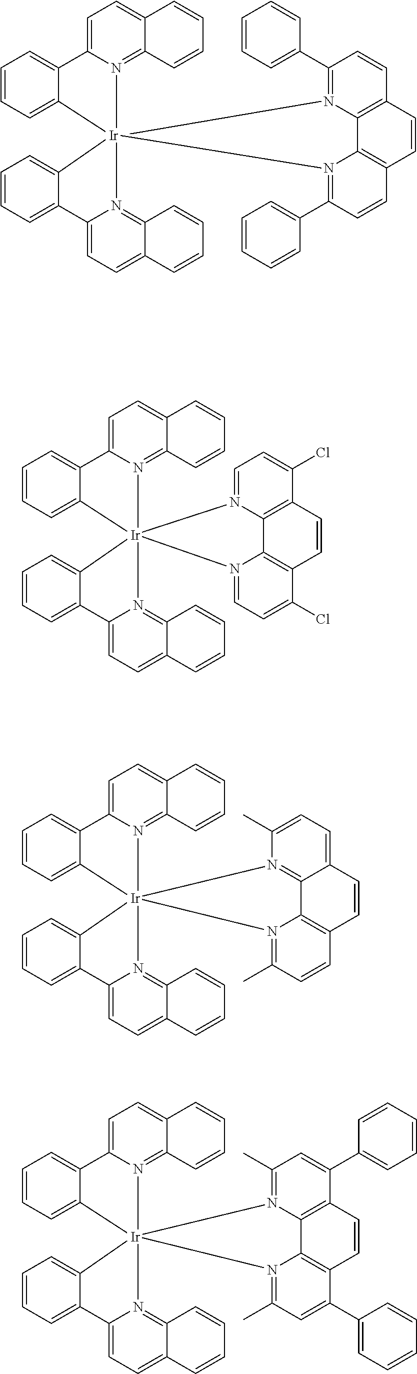

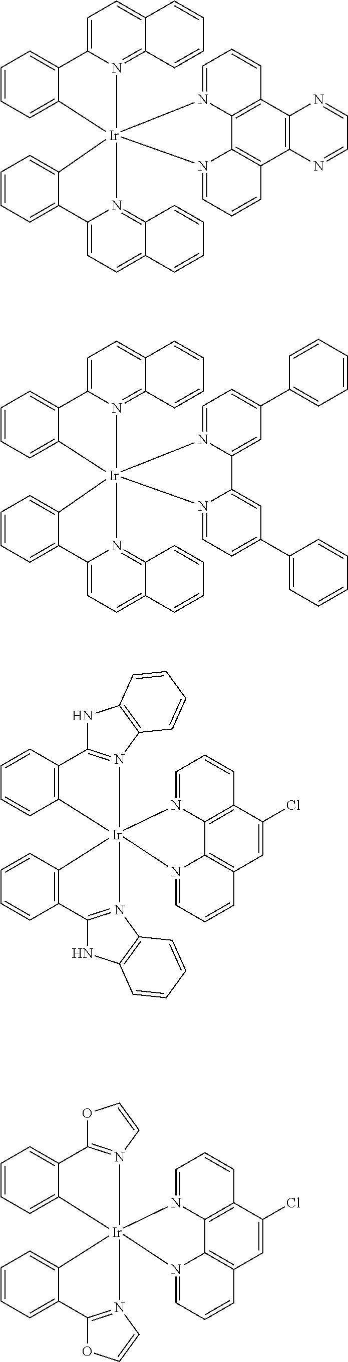

In accordance with a third aspect of the present invention, there is provided a method of screening a luminescent probe for detecting helicase activity, comprising the steps of: (i) measuring luminescent responses of a luminescent probe candidate towards a G-quadruplex DNA, a double-stranded DNA, and a single-stranded DNA respectively; and (ii) determining selectivity of the luminescent probe candidate towards the G-quadruplex DNA over the double-stranded DNA, and the single-stranded DNA based on the luminescent responses measured in step (i).

Preferably, the helicase is capable of unwinding the double-stranded DNA to provide the single-stranded DNA and a G-quadruplex-forming DNA in which the G-quadruplex-forming DNA folds to form the G-quadruplex DNA.

It is preferable that the G-quadruplex DNA comprises a sequence of SEQ ID NO: 1.

Advantageously, the selectivity of the luminescent probe candidate towards the G-quadruplex DNA over the double-stranded DNA is determined if a ratio between the luminescent response of the luminescent probe candidate towards the G-quadruplex DNA and that towards the double-stranded DNA is larger than 1; and the selectivity of the luminescent probe candidate towards the G-quadruplex DNA over the single-stranded DNA is determined if a ratio between the luminescent response of the luminescent probe candidate towards the G-quadruplex DNA and that towards the single-stranded DNA is larger than 1.

Preferably, the helicase is a hepatitis C virus NS3 helicase.

More preferably, the method of the third aspect further comprises the step of (iii) measuring a luminescent response of the luminescent probe candidate towards the helicase.

Advantageously, the selectivity of the luminescent probe candidate towards the G-quadruplex DNA over the helicase is determined if a ratio between the luminescent response of the luminescent probe candidate towards the G-quadruplex DNA and that towards the helicase is larger than 1.

Accordingly, the present invention provides a novel and highly advantageous approach to detect helicase activity in a test solution, namely by using a luminescent probe such as a Ir(III) complex. In addition, a series of luminescent Ir(III) complexes proved to achieve prominent effect on detecting the presence of G-quadruplex DNA so as to generate luminescent responses for detection of helicase activity.

### BRIEF DESCRIPTION OF THE DRAWINGS

The above and other objects and features of the present invention will become apparent from the following description of the invention, when taken in conjunction with the accompanying drawings, in which:

FIG. 1 shows a schematic diagram of the luminescent switch-on assay to monitor the duplex-DNA unwinding activity of helicase using a G-quadruplex-selective probe.

FIG. 2 shows chemical structures of the luminescent Ir(III) complexes 1-17 that were synthesised and evaluated.

FIG. 3a shows the chemical structure of a preferred Ir(III) complex of the present invention, complex 9.

FIG. 3b shows G4-FID titration curves of DNA duplex ds17 and Pu27 G-quadruplex in the presence of increasing concentration of complex 9 in Tris-HCl buffer, wherein DC₅₀ value is determined by the half-maximal concentration of compound required to displace 50% TO from DNA.

FIG. 4a shows luminescence spectra of the complex 9+G4-quadruplex system in response to various concentrations of helicase: 0, 0.09, 0.18, 0.27, 0.36, 0.45, 0.54, 0.72, and 0.9 μM.

FIG. 4b shows the relationship between luminescence intensity at λ=571 nm and concentration of helicase; wherein the insert shows a linear plot of the change in luminescence intensity at λ=571 nm vs. concentration of helicase.

FIG. 5a shows luminescence response of the complex 9+G4-quadruplex system with helicase or S1, Endo, DpnI, ExoI, EcoRI, RNase, DNase and SSB.

FIG. 5b shows luminescence spectra of the complex 9+G-quadruplex system in a reaction system containing 0.5% (v/v) cell extract in response to various concentrations of helicase: 0, 0.18, 0.36, 0.45, 0.54, 0.72, and 0.9 μM.

FIG. 5c shows relative luminescence intensity of the system in the presence of different concentrations of ciprofloxacin: 0, 1, 2.5, 5, 10, and 20 μM.

FIG. 5d shows emission spectra of complex 9 in the absence of ciprofloxacin (0 μM) and in the presence of ciprofloxacin (20 μM).

FIG. 5e shows relative luminescence response of the complex 9+G-quadruplex ensemble upon the addition of 20 μM ciprofloxacin.

FIG. 5f shows luminescence response of the complex 9+G-quadruplex systems treated with 10 μM of suramin, TBBT or ciprofloxacin in the presence of 0.8 μM helicase, respectively, compared with the control group treated with 0.8μM helicase only.

FIG. 6 shows a diagrammatic bar array representation of the luminescence enhancement selectivity ratio (I/I₀) of complexes 1-7 for PS2. M G-quadruplex DNA (G4) over dsDNA (ds17) and ssDNA (CCRS-DEL), in particular I_{G4}/I_{dsDNA} refers to the ratio of the luminescent response of PS2. M G-quadruplex DNA over dsDNA (ds17); and I_{G4}/I_{ssDNA} refers to the ratio of the luminescent response of PS2. M G-quadruplex DNA over ssDNA(CCR5-DEL).

FIG. 7 shows a diagrammatic bar array representation of the luminescence enhancement selectivity ratio (I/I₀) of complexes 7-17 for PS2. M G-quadruplex DNA over dsDNA (ds17) and ssDNA (CCRS-DEL), in particular I_{G4}/I_{dsDNA} refers to the ratio of the luminescent response of PS2. M G-quadruplex DNA over dsDNA (ds17); and I_{G4}/I_{ssDNA} refers to the ratio of the luminescent response of PS2. M G-quadruplex DNA over ssDNA(CCR5-DEL).

FIG. 8a shows a melting profile of F21T G-quadruplex DNA (0.2 μM) in the absence of complex 9 (0 μM) and in the presence of complex 9 (5 μM).

FIG. 8b shows a melting profile of F10T dsDNA (0.2 μM) in the absence of complex 9 (0 μM) and in the presence of complex 9 (5 μM).

FIG. 9 shows the emission spectrum of the system with complex 9 alone (the concentration of the complex 9 is 1 μM) in the absence of helicase (indicated as grey line) and in the presence of helicase (0.9 μM) (indicated as black line) respectively.

FIG. 10 shows the emission spectrum of complex 9 (1 μM) in the presence of helicase (0.9 μM) and ON1ₘ/ON2 duplex mutant (0.25 μM).

FIG. 11 shows the relative luminescence response of complex 9+G-quadruplex ensemble upon the addition of 0.8 μM HCV NS3 helicase.

FIG. 12 shows the relative luminescence response of the system in the presence of helicase (0.9 μM) at various concentrations of complex 9 (0.25, 0.5, 1 and 2 μM), wherein 1 μM of complex 9 offered the highest luminescence fold-change response compared to 0.25, 0.5 or 2 μM of complex 9.

FIG. 13 shows the relative luminescence response of the system in the presence of helicase (0.9 μM) at various concentrations of duplex DNA (0.125, 0.25, 0.5, and 1 μM), and it was observed that the luminescence response of the system was highest luminance at 0.25 μM concentration of duplex DNA.

FIG. 14 shows the relative luminescence response of the system in the presence of helicase (0.9 μM) at various concentrations of ATP (0.2, 0.5, 1, and 2.5 mM), it was observed that the luminescence response of the system was highest luminance at 1 mM concentration of ATP.

FIG. 15 shows the emission spectrum of complex 9 (1 μM) and duplex DNA (0.25 μM) upon incubation with helicase (0.09 μM) in Tris-HCl buffer (20 mM, 50 mM KCl, 150 mM NH₄Ac, pH 7.2) (indicated as black line), and without incubation with helicase (indicated as grey line).

FIG. 16a shows relative luminescence response of complex 9 upon the addition of 10 μM of suramin or TBBT.

FIG. 16b shows relative luminescence response of the complex 9+G-quadruplex ensemble upon the addition of 10 μM of suramin or TBBT.

FIG. 17 shows a diagrammatic bar array representation of the luminescence enhancement selectivity ratio of complexes 1, 2, 4, and 7-13 for PS2. M G-quadruplex DNA over helicase, I_{G4}/I_{helicase} refers to the ratio of the lunminscent response of PS2. M G-quadruplex DNA over helicase.

### DETAILED DESCRIPTION OF THE INVENTION

The present invention, in a first aspect, relates to a method of detecting helicase activity in a test solution. Said method comprises the steps of: (a) providing a substrate solution containing a double-stranded DNA, wherein said double-stranded DNA is a substrate for a helicase to be detected; (b) introducing the substrate solution to the test solution to form a reaction solution; (c) applying a luminescent probe into the reaction solution to form a mixture; and (d) measuring a luminescent response of the luminescent probe in the mixture, wherein the luminescent response corresponds to the helicase activity in the test solution.

The term “helicase” as used in the present invention generally refers to a class of enzymes which is capable of separating two annealed nucleic acid strands such as double-stranded DNA, self-annealed RNA and RNA-DNA hybrid. Preferably, helicase is β helicase that works on double-stranded DNAs. In preferred embodiments, the helicase is virus helicase such as hepatitis virus helicase. In a most preferred embodiment, the helicase is hepatitis C virus NS3 helicase. The helicase activity in the present invention preferably refers to the unwinding activity of the helicase on nucleic acids strands. The expressions “oligonucleotide” and “oligomer” as used in this application generally refer to short, single-stranded nucleic acid fragments, e.g. short DNA fragments. The skilled person would understand that “double-stranded oligonucleotide” refers to “double-stranded DNA”.

The method of the present invention provides a double-stranded DNA which can be unwound by the helicase to be detected. The double-stranded DNA may be obtained from natural source or obtained by artificial creation. The double-stranded DNA is preferably provided in a form of solution containing a buffer solution to form a substrate solution, wherein the buffer solution prevents substantial change in the pH. Preferably, the double-stranded DNA (dsDNA) is unwound by the helicase under suitable conditions to provide two single-stranded DNAs (ssDNAs), for example, the reaction solution is heated to a temperature of about 37° C. for at least 30 minutes, preferably 2 hours, to allow the helicase to unwind the double-stranded DNA.

In preferred embodiments of the present invention, one of the ssDNAs is a guanine-rich (G-rich) DNA, i.e. G-quadruplex-forming DNA which further folds to form a G-quadruplex DNA, and another one is a cytosine-rich (C-rich) DNA being a complementary sequence to the G-rich sequence. More preferably, the G-quadruplex-forming DNA includes a sequence of SEQ ID NO: 1. Said sequence enables the G-quadruplex-forming DNA to fold under conditions, namely in the presence of cations such as potassium ions (K⁺).

The formed G-quadruplex DNA of the present invention can interact with the luminescent probe to generate a luminescent response for detection. Preferably, the luminescent probe binds to the G-quadruplex DNA with molecular interactions. In particular, such binding is of advantageous that it suppresses the non-radiative decay of the excited state of the luminescent probe and allows an enhanced luminescent response such as improved quantum yield and longer lifetime. The detection of the luminescent response, i.e. step (d), may be carried out by using UV/Vis absorption spectrometer, depending on the emission wavelength of the luminescent probe. The skilled person would appreciate that other common means can also be applied for measuring the luminescent response. In some preferred embodiments, the measuring step in step (d) of the method includes steps of exciting the mixture with a radiating source at a wavelength of about 360 nm, and recording the results of said excitation at emission wavelengths range between 500-720 nm.

In preferred embodiments, the luminescent probe is a luminescent iridium (III) (Ir(III)) complex. In particular, the Ir(III) complex, as will be discussed in more detail later, comprises an iridium-containing dimer; and a nitrogen-containing ligand having at least two nitrogen atoms, wherein the nitrogen-containing ligand is a derivative of pyridine.

In some embodiments, the test solution may be a biological sample or chemical sample, preferably a biological sample. The biological sample, without limitation, refers to any sample which comprises cells or cellular material which may be obtained in vivo or in vitro.

In further embodiments, the method of detecting helicase activity further comprises the steps of adding a quenching reagent, e.g. ethylenediaminetetraacetic acid (EDTA), to the reaction solution after step (b) to quench the unwinding activity of the helicase and, preferably, subsequently diluting the reaction solution to a suitable concentration prior to step (c). Preferably, the reaction solution is diluted by adding a buffer solution. In particular, said dilution allows the helicase to have a concentration of 0 to 0.9 μM in the reaction solution so as to facilitate the detection of helicase activity as it has been surprisingly found that the luminescence responses of the helicase of said concentration range in the solution present a linear range of detection.

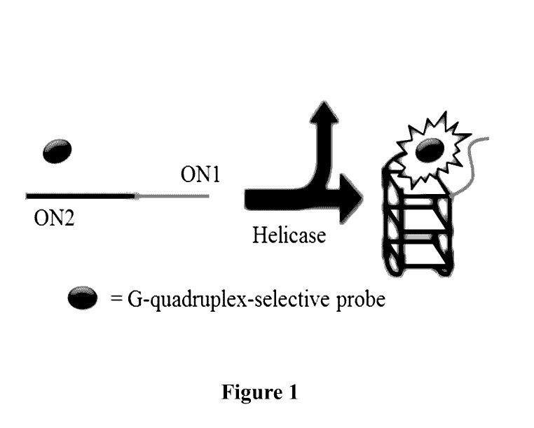

With reference to FIG. 1, there is a schematic diagram showing the proposed mechanism of the method of detecting helicase activity of the present invention. In a preferred embodiment, there is provided a designed double-stranded oligomer consisting of a G-quadruplex-forming sequence, i.e. guanine-rich sequence, (ON1, corresponding to SEQ ID NO: 2) and its complementary cytosine-rich sequence (ON2, corresponding to SEQ ID NO: 3), which acts as a substrate for a helicase in particular hepatitis C virus (HCV) NS3 helicase. In the absence of helicase, the double-stranded oligonucleotide substrate remains as a duplex structure that interacts only weakly with a luminescent probe such as a luminescent Ir(III) complex. However, in the presence of helicase, the helicase unwinds the duplex DNA substrate and generates two ssDNA fragments. When a quenching reagent EDTA is added to stop the reaction between the helicase and the duplex substrate, the G-quadruplex-forming oligomer ON1 folds into a G-quadruplex motif in the presence of K⁺ ions. The nascent G-quadruplex structure is then recognized by the luminescent Ir(III) complex, as indicated as a G-quadruplex-selective probe in FIG. 1, with an enhanced emission response. As such, the present method is capable of functioning as a switch-on luminescent probe for helicase activity. In turn, the G-quadruplex structure also allows for screening of a potent luminescent probe.

Luminescent Ir(III) Complexes

In general, luminescent transition metal complexes have notable advantages over organic dyes for sensory applications. Firstly, metal complexes generally emit in the visible region with a long phosphorescence lifetime, allowing them to be readily distinguished from a fluorescent background arising from endogenous fluorophores in the sample matrix by the use of time-resolved fluorescent spectroscopy. Secondly, the precise and versatile arrangement of co-ligands on the metal centre allows the interactions of metal complexes with biomolecules to be fine-tuned for maximum selectivity and sensitivity. Thirdly, these metal complexes often possess interesting photophysical properties that are strongly affected by subtle changes in their local environment. In this regard, the present invention further provides a series of luminescent transition metals such as Ir(III) complexes to establish a label-free, sensitive and efficient assay on detecting helicase activity.

In a second aspect of the present invention, there is provided with a luminescent Ir(III) complex having an iridium-containing dimer and a nitrogen-containing ligand with at least two nitrogen atoms. In preferred embodiments, the luminescent Ir(III) complex includes an iridium-containing dimer having at least two benzene rings; and a nitrogen-containing ligand (N̂N ligand) which is a derivative of pyridine. In particular, the iridium-containing dimer includes at least two carbon-nitrogen-containing ligands (ĈN ligand) and each of them bearing a benzene ring.

In some preferred embodiments, the ĈN ligand has a chemical structure selected from the group consisting of the following structures:

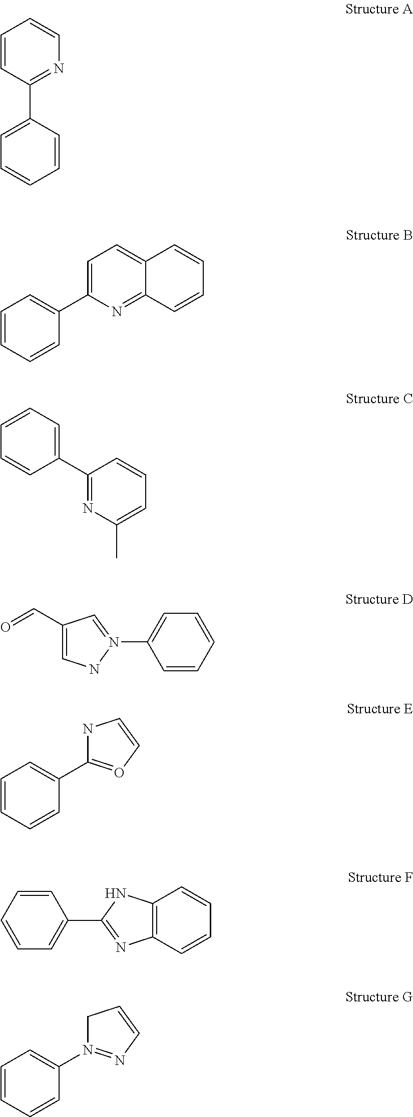

In some preferred embodiments, the nitrogen-containing ligand (N̂N ligand) has at least two nitrogen atoms and, in particular, selected from the group consisting of 1,10-phenanthroline (phen), 2,9-diphenyl-1,10-phenanthroline (2,9-dpphen), 2,9-dimethyl-4,7-diphenyl-1,10-phenanthroline (dmdpphen), 5,6-dimethyl-1,10-phenanthroline (dmphen), 5-chloro-1,10-phenanthroline (chlorophen), 4,7-dichloro-1,10-phenanthroline (dcphen), 2,2′-bipyridine (bpy), 5,5′-dimethyl-2,2′-bipyridine (5,5-dmbpy), 4,7-diphenyl-1,10-phenanthroline (4,7-dpphen), 4,4′-diphenyl-2,2′-bipyridine (dpbpy), and pyrazino[2,3-f][1,10]phenanthroline (pyphen). The chemical structures of the above N̂N ligands are shown below.

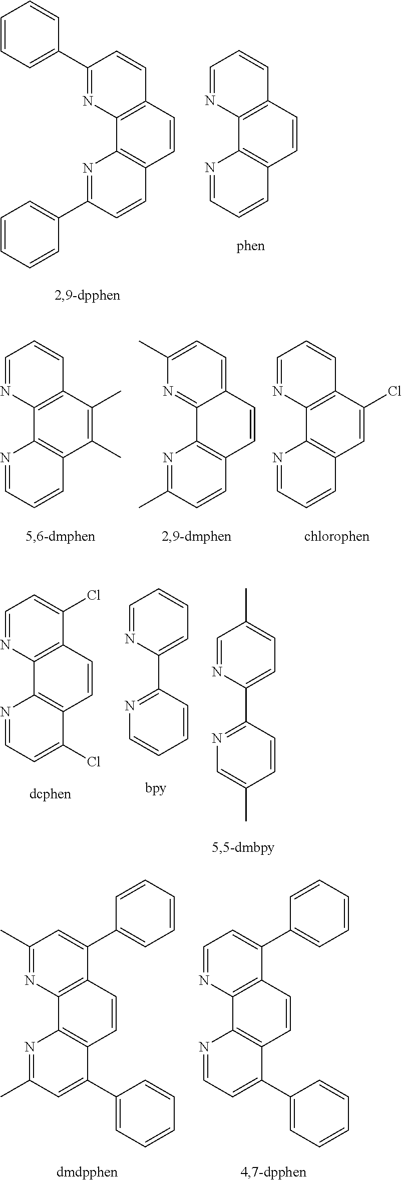

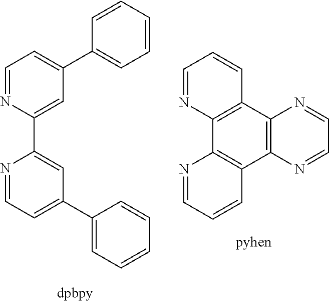

Some specific examples of the preferred luminescent Ir(III) complexes include those having one of the following structures:

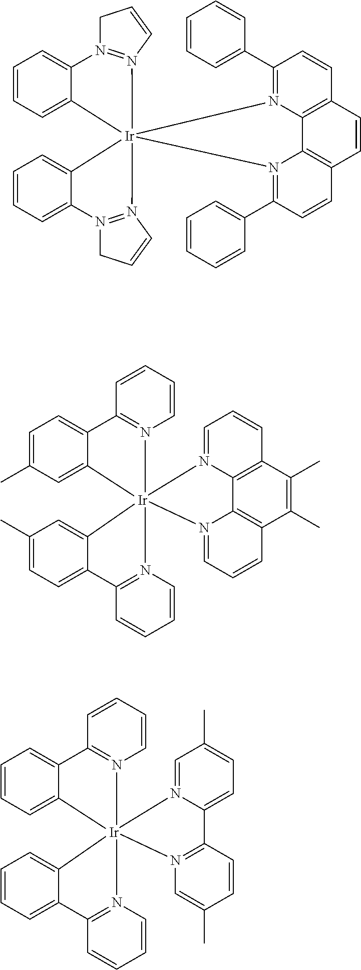

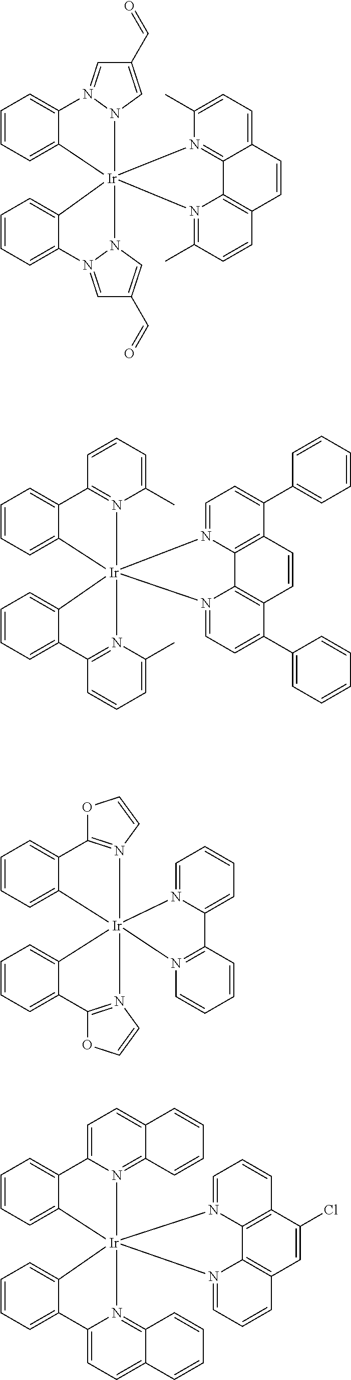

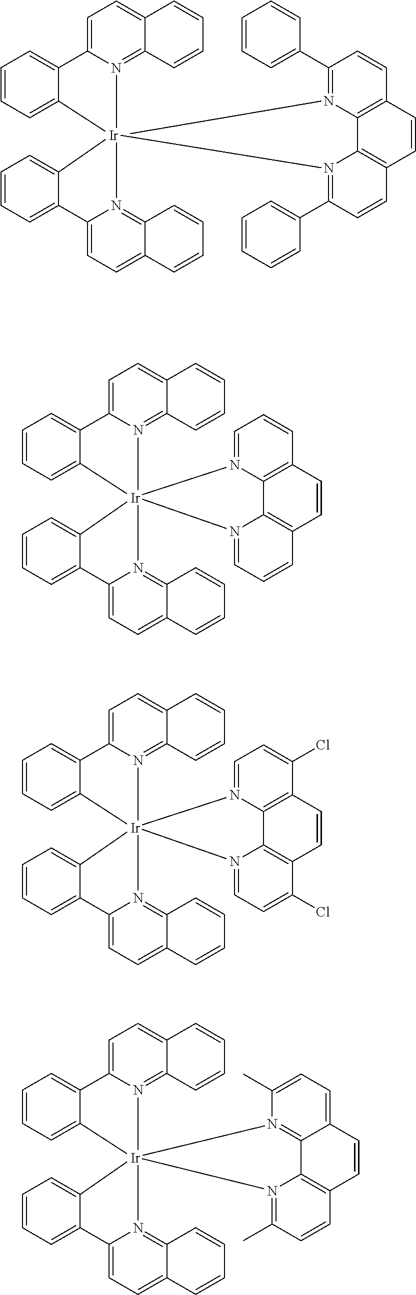

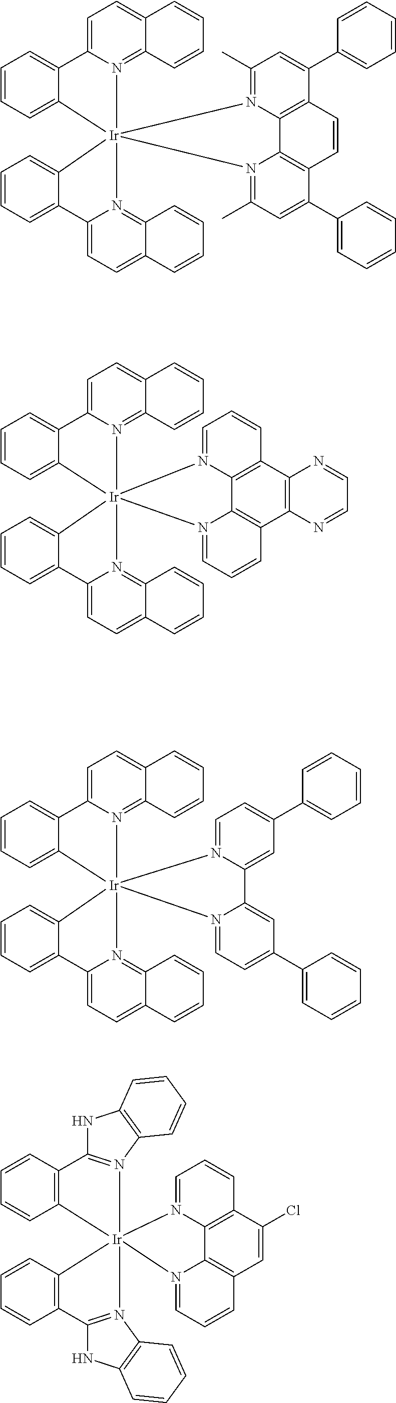

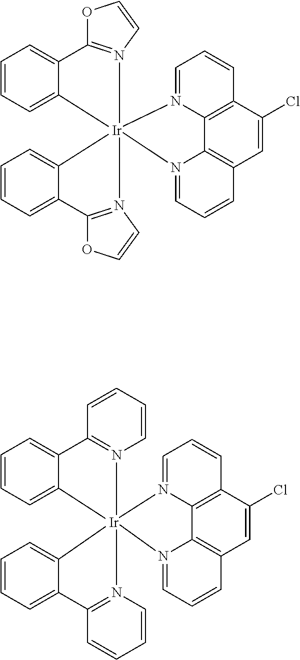

Screening G-Quadruplex-Selective Probes

In a further aspect of the present invention, the G-quadruplex DNA is used to identify a potent luminescent probe for detecting the respective helicase activity. The potent luminescent probe is a luminescent compound that can selectively recognize the G-quadruplex DNA to give the corresponding luminescent response. The potent luminescent probe may also be applied to determine the unknown helicase activity. In order to determine the selectivity of the luminescent compound towards the G-quadruplex DNA, the luminescent responses of the luminescent compound towards the G-quadruplex DNA, dsDNA, ssDNA, as well as the respective helicase are considered.

In some preferred embodiments, there are provided with various luminescent probe candidates. The luminescent responses of the luminescent probe candidates towards each of the G-quadruplex DNA, dsDNA, ssDNA, and the respective helicase are measured. Preferably, the selectivity of the luminescent probe candidate towards the G-quadruplex DNA is determined if:
(i) a ratio between the luminescent response of the luminescent probe candidate towards the G-quadruplex DNA and that towards the double-stranded DNA is larger than 1; and/or
(ii) a ratio between the luminescent response of the luminescent probe candidate towards the G-quadruplex DNA and that towards the single-stranded DNA is larger than 1; and/or
(iii) if a ratio between the luminescent response of the luminescent probe candidate towards the G-quadruplex DNA and that towards the helicase is larger than 1.

More preferably, the potent luminescent probe at least has a selectivity towards the G-quadruplex DNA over the dsDNA and ssDNA.

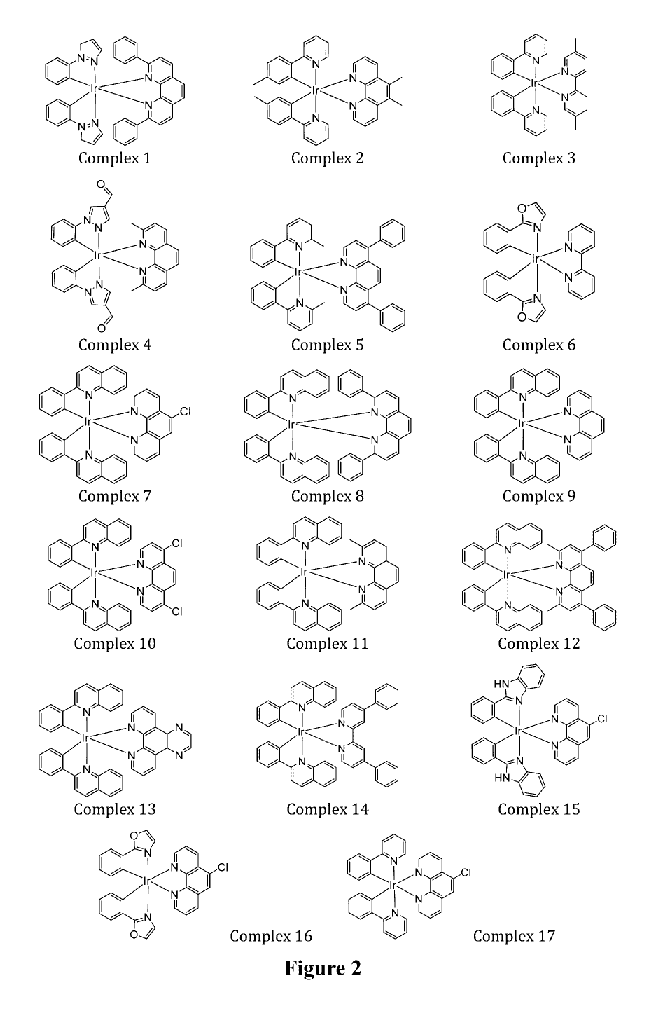

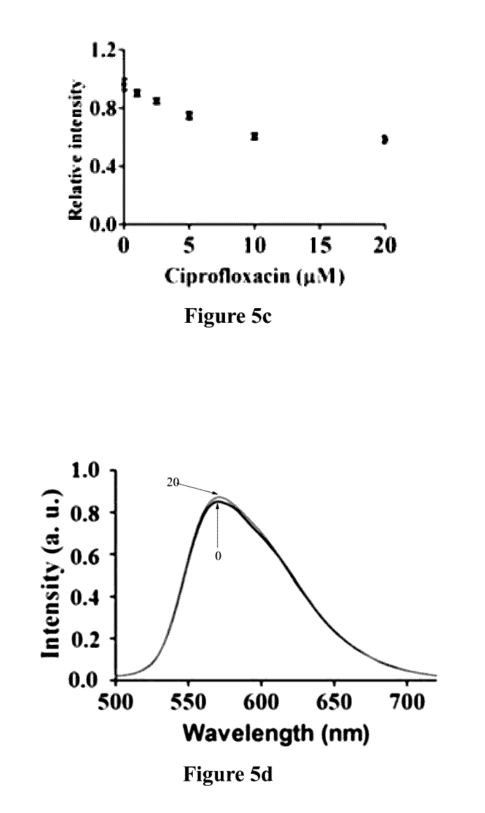

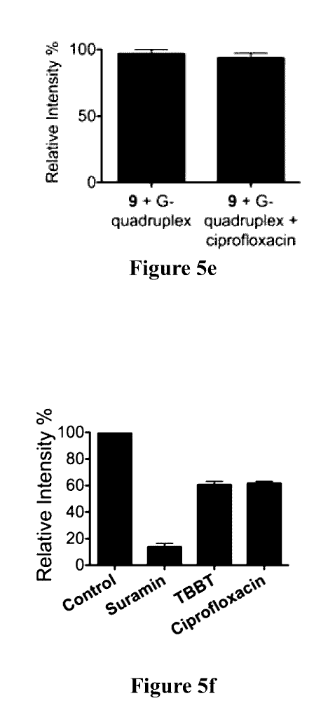

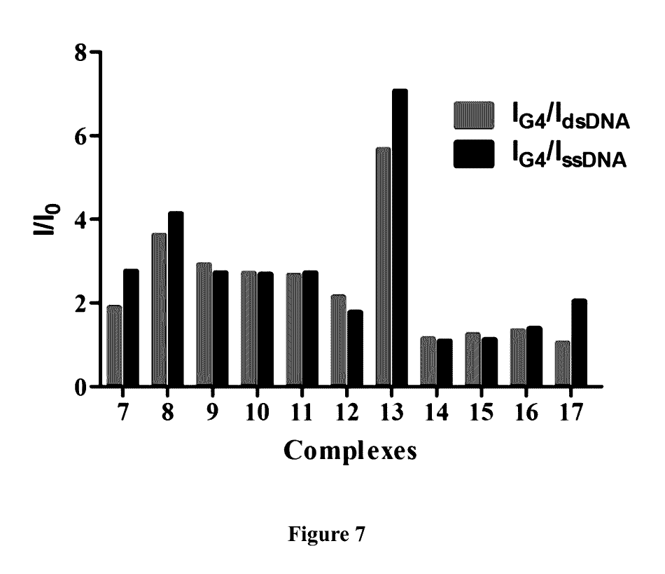

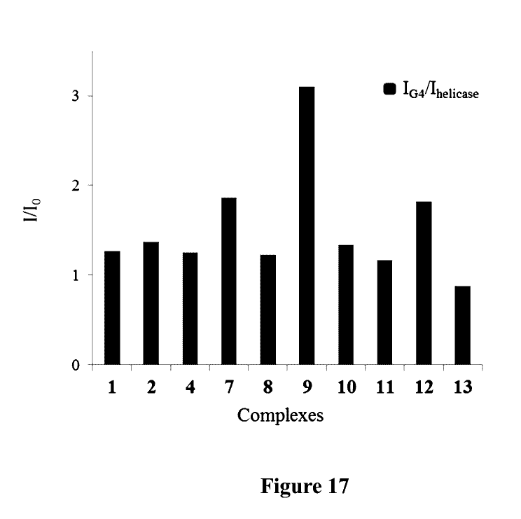

In one embodiment of the present invention, seven luminescent Ir(III) complexes 1-7, as shown in FIG. 2 were initially examined for their emission response to different forms of DNA, including G-quadruplex, ssDNA and dsDNA (Table 1). With reference to FIG. 6, of these seven complexes, only complex 7 bearing the N̂N ligand chlorophen (5-chloro-1,10-phenanthroline) and the ĈN ligand phq showed a selective response for G-quadruplex DNA, while not showing any luminescence enhancement towards helicase (FIG. 17). On the other hand, complexes 3, 5, and 6 were found to be non-selective for G-quadruplex DNA. Also, with reference to FIG. 17, the luminescence of complexes 1, 2 and 4 were enhanced in the presence of helicase only. Based on the structure of complex 7, a focused library of eleven Ir(III) complexes (7-17, FIG. 1) containing phq and chlorophen derivatives as ligands were designed and synthesised. The second round of screening revealed that the complexes 14-17 were non-selective for G-quadruplex DNA (FIG. 7). The luminescence of complexes 8, and 10-13 were enhanced with addition of helicase only and the respective selectivity towards G-quadruplex DNA over helicase is relatively low when compared with complex 9, as shown in FIG. 17. After excluding complexes 8, and 10-13, the Ir(III) complex 9 displayed a significantly enhanced luminescence response in the presence of the PS2.M G-quadruplex (FIG. 7), and no luminescence enhancement in the presence of helicase (FIG. 9) indicating that complex 9 did not directly interact with helicase.

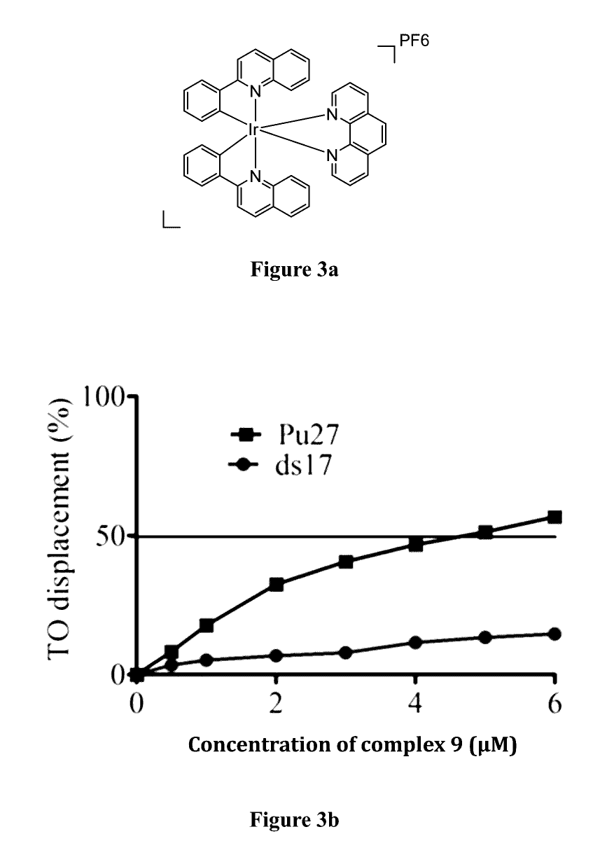

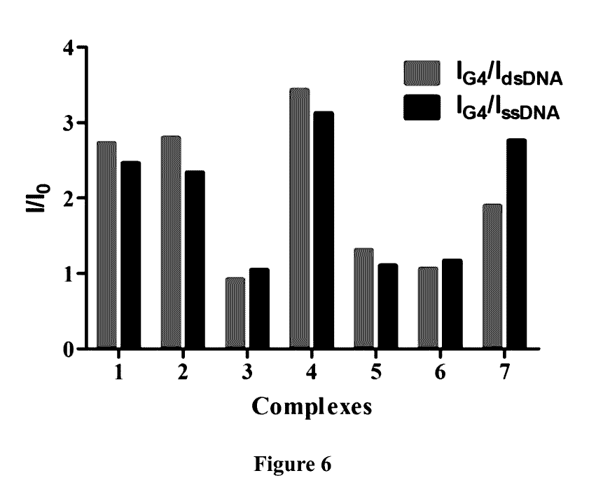

To further validate the suitability of complex 9 as a G-quadruplex-selective probe, the inventors performed G-quadruplex fluorescent intercalator displacement (G4-FID) and fluorescence resonance energy transfer (FRET) melting assays to determine the selectivity of complex 9 for G-quadruplex DNA. The G4-FID assay also showed that complex 9 was able to displace thiazole orange (TO) from G-quadruplex DNA (^{G4}DC₅₀=ca. 5 μM, half-maximal concentration of compound required to displace 50% TO from DNA) with higher efficacy than from duplex DNA (FIG. 3b). Additionally, FRET-melting assays revealed that the melting temperature (ΔTₘ) of the F21T G-quadruplex was increased by about 13° C. upon the addition of complex 9 (FIG. 8a). By comparison, only 4° C. change in the melting temperature of F10T dsDNA was observed at the same concentration of complex 9 (FIG. 8b). Taken together, these results demonstrate the ability of complex 9 to discriminate between G-quadruplex DNA and dsDNA or ssDNA. The luminescence enhancement of complex 9 in the presence of G-quadruplex DNA is presumably due to its ability to bind to G-quadruplex structures through groove/loop binding or end-stacking interactions. This shields the complex from the aqueous solvent environment and suppresses non-radiative decay of the excited state, thus leading to enhanced triplet state emission.

Luminescent Detection of HCV NS3 Helicase Activity, and Optimization

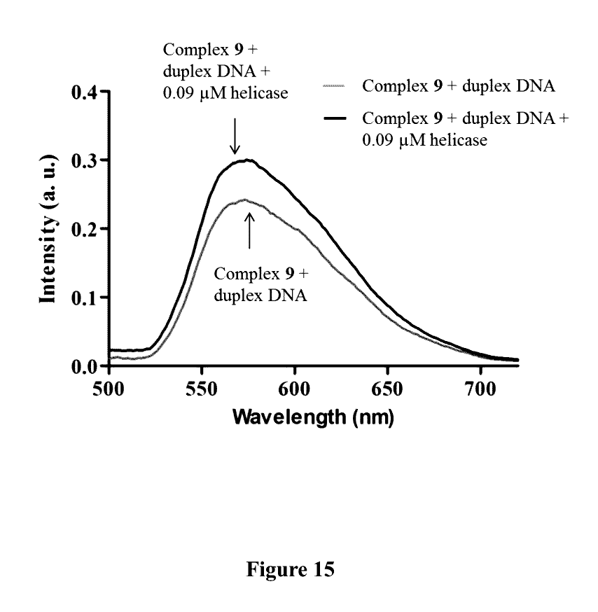

The characterization and photophysical properties of the Ir(III) complexes 1-17 are given in the ESI (Table 2). Given the promising G-quadruplex-selective luminescent behaviour exhibited by complex 9, one preferred embodiment of the present invention sought to employ complex 9 as a G-quadruplex-selective probe to construct a label-free luminescent detection platform for helicase activity in aqueous solution. This embodiment first investigated the luminescence response of complex 9 and the ON1-ON2 duplex substrate to helicase. Upon incubation with helicase and the duplex substrate, the luminescence of complex 9 was significantly enhanced. The luminescence enhancement of the system was possibly due to the unwinding of the duplex substrate by helicase, which allows the formation of the G-quadruplex motif in the presence of K⁺ that is subsequently recognized by complex 9 (FIG. 15).

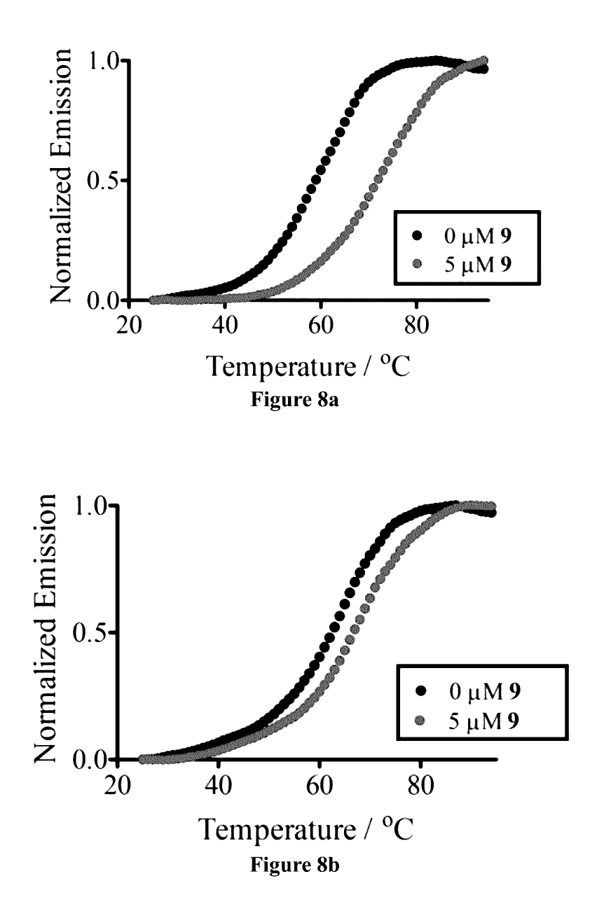

Further control experiments were conducted. In particular, a designed mutant DNA sequence (ON1ₘ, corresponding to SEQ ID NO: 4), which is unable to form a G-quadruplex structure in the presence of helicase due to the lack of guanine residues, was also used to confirm the action of complex 9. A slight decrease was observed in the luminescence of complex 9 in response to helicase for the mutant DNA sequences, indicating that the formation of the G-quadruplex motif was important for the luminescent enhancement of the system (FIG. 10). Taken together, these results suggest that the luminescence enhancement of the system originated from the specific interaction of complex 9 with the G-quadruplex motif, which is generated by the unwinding of the duplex DNA substrate by helicase.

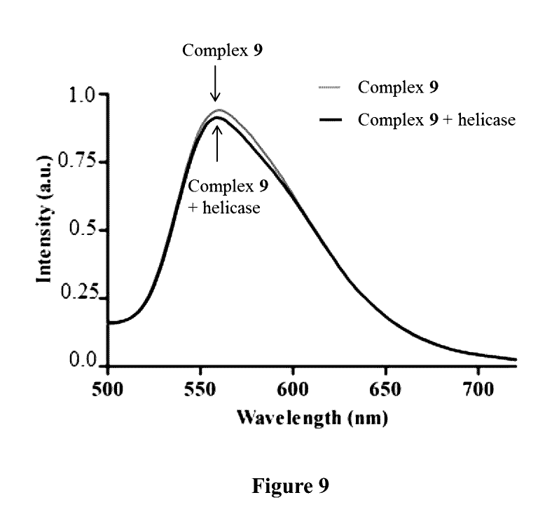

Various studies have been performed to investigate the ability of helicases to unfold G-quadruplex structures. One study in J. B. Budhathoki, S. Ray, V. Urban, P. Janscak, J. G. Yodh and H. Balci, Nucleic Acids Res., 2014, 42, 11528-11545 reported that Bloom's syndrome helicase (BLM) could unfold telomeric G-quadruplex in the absence of ATP, while another study in J.-q. Liu, C.-y. Chen, Y. Xue, Y.-h. Hao and Z. Tan, J. Am. Chem. Soc., 2010, 132, 10521-10527 reported that BLM translocation was hindered by G-quadruplex motifs, with unwinding efficiency being dependent on the stability of the G-quadruplex structure, which is in turn influenced by loop size or ionic strength. For example, the unfolding activity of BLM towards a particular G-quadruplex sequence was completely stopped in 150 mM K⁺. Therefore, it was important to investigate whether HCV NS3 helicase could unfold the G-quadruplex structure used in the present invention. With reference to FIG. 11, the results showed that no significant change in the luminescence intensity of the complex 9+G-quadruplex ensemble was observed upon the addition of 0.8 μM HCV NS3 helicase, indicating that this helicase did not unfold the G-quadruplex structure employed in this study. A person skilled in the art would understand that optimization may be required for other helicases by using any common methods in the art.

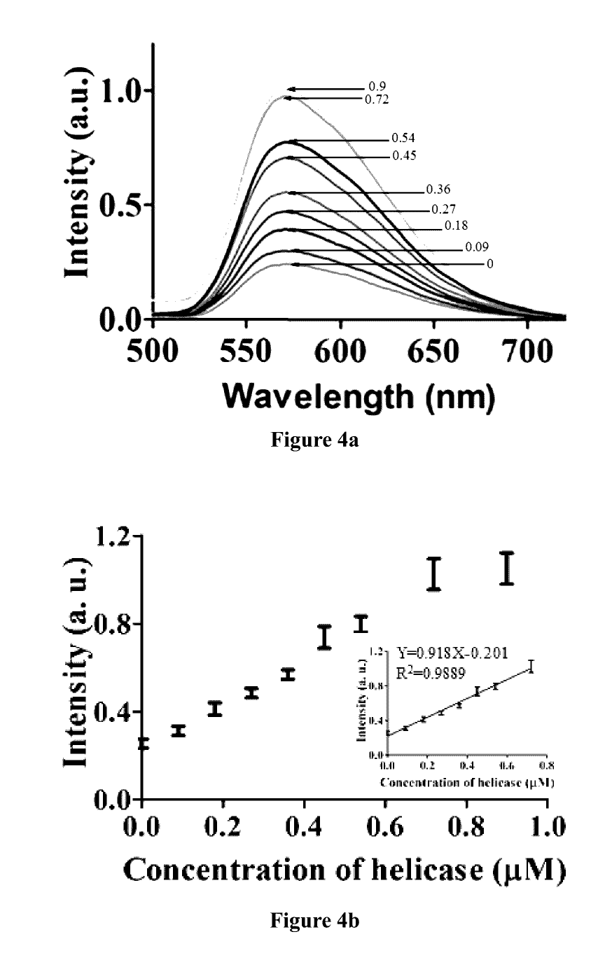

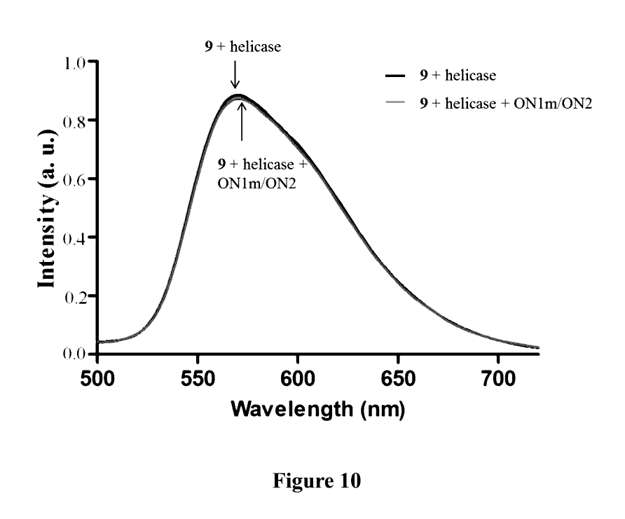

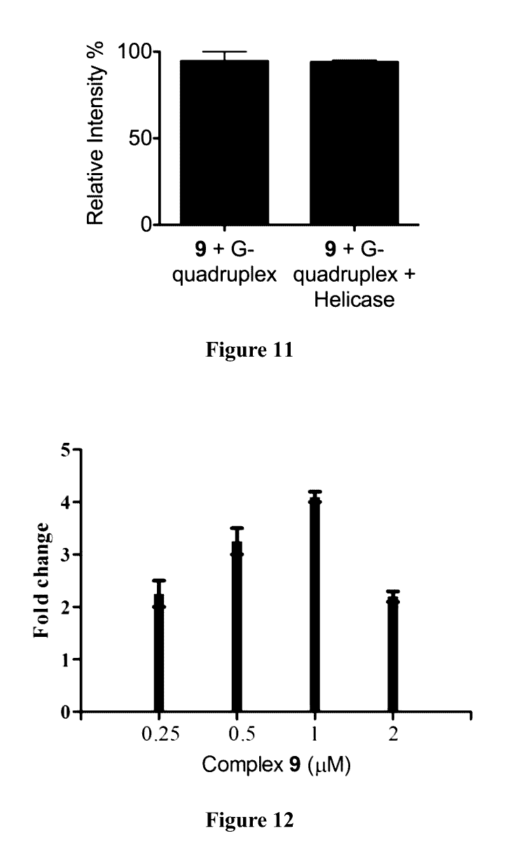

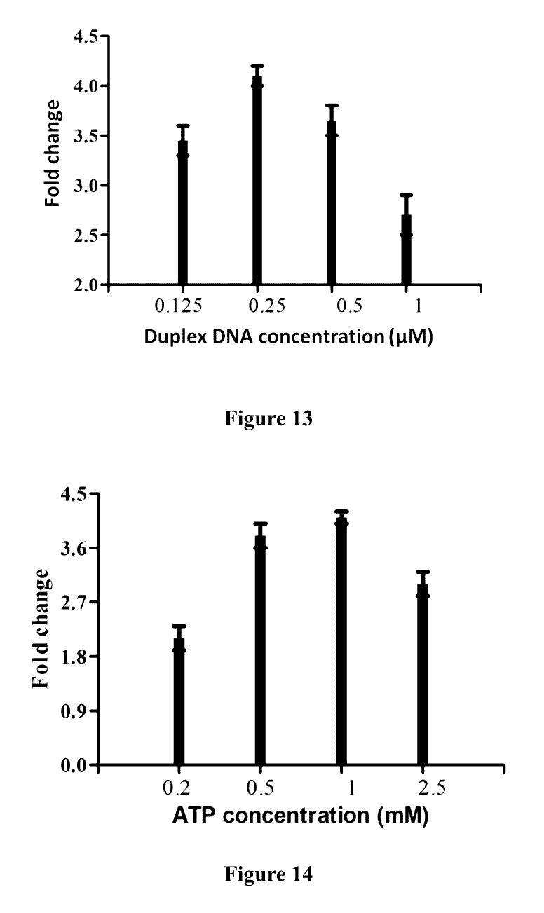

After optimization of the concentrations of complex 9 (FIG. 12), DNA (FIG. 13) and ATP (FIG. 14), one embodiment of the present invention investigated the luminescence response of the system to different concentrations of helicase. The system exhibited a ca. 4.5-fold enhancement in luminescence when the helicase concentration was 0.9 μM (FIG. 4a), with a linear range of detection for helicase from 0 to 0.72 μM (FIG. 4b). Furthermore, the detection limit of this assay for helicase was estimated to be 0.09 μM ata signal-to-noise ratio (S/N) of 3 (FIGS. 4b and 15).

Selectivity of G-Quadruplex-Based HCV NS3 Helicase Activity Assay

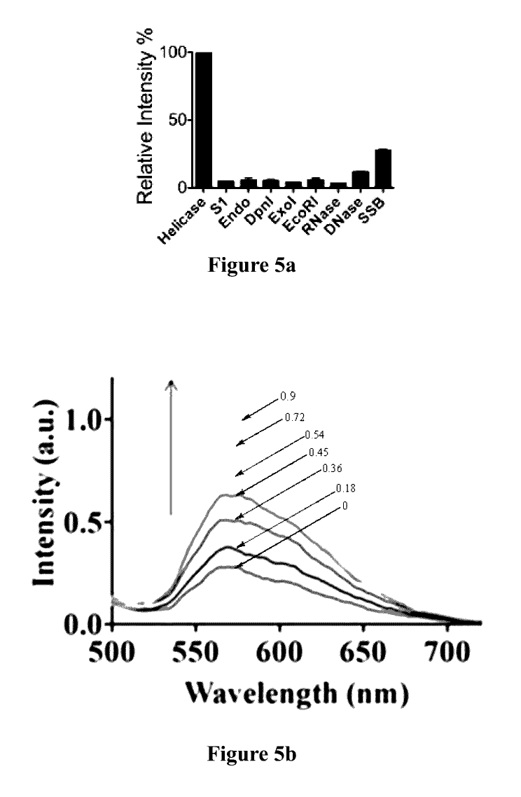

In a specific preferred embodiment, the selectivity of HCV NS3 helicase activity assay in the present invention was evaluated by investigating the response of the system to S1 nuclease (S1), endonuclease IV (Endo), DpnI, exonuclease I (ExoI), EcoRI, RNase, DNase or single-stranded DNA binding protein (SSB). With reference to FIG. 5a, the results showed that only helicase could significantly enhance the luminescence of the complex 9+G-quadruplex DNA system. No significant change in emission intensity was observed upon the addition of the nucleases, while a relatively low emission enhancement was observed for single-stranded DNA binding protein. These results indicate that the system displays selectivity for helicase over nucleases or single-stranded DNA binding proteins, which originates presumably from unwinding of the duplex DNA substrate by helicase.

Application of HCV NS3 Helicase Activity Detection Assay in Biological Samples

To evaluate the robustness of the system, the performance of G-quadruplex-based sensing platform for helicase activity of the present invention was investigated in the presence of cellular debris. According to FIG. 5b, in a reaction system containing 0.5% (v/v) cell extract, the complex 9+G-quadruplex DNA system experienced a gradual increase in luminescence intensity as the concentration of helicase was increased. Accordingly, the present invention provides a useful method of determining helicase unwinding activity in biological samples.

Application of HCV NS3 Helicase Activity Detection Assay in Inhibitors Screening

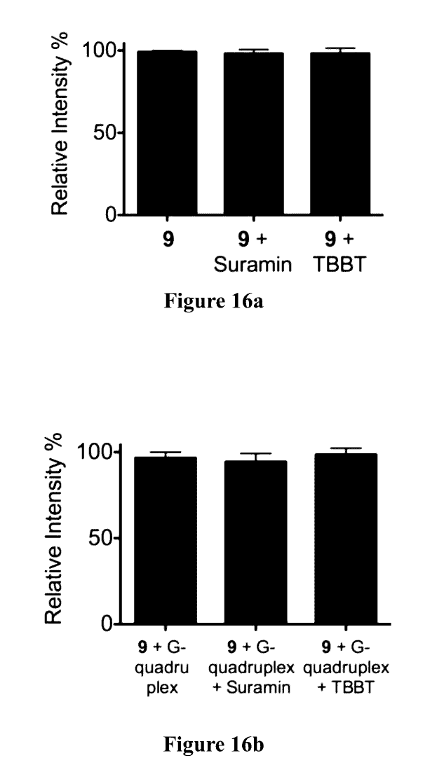

In one embodiment of the present invention, the G-quadruplex-based assay is applied for screening potential helicase inhibitors. In particular, ciprofloxacin was chosen as an inhibitor of helicase. With reference to FIG. 5c, the luminescence signal of the system was diminished in the presence of ciprofloxacin in a dose-dependent manner, with a decrease of about 40% observed at 10 μM of ciprofloxacin. Ciprofloxacin has no direct quenching effect on the luminescence of complex 9, as shown in FIG. 5d, or the complex 9+G-quadruplex ensemble, as shown in FIG. 5e. Accordingly, the method of the present invention is capable of screening potential helicase inhibitors and is beneficial to a high-throughput screening. To further demonstrate the application of the G-quadruplex-based assay for inhibitor screening, a group of well-known HCV NS3 helicase inhibitors was investigated. The tested inhibitors, suramin and TBBT, displayed inhibitory activity towards HCV NS3 helicase in this platform, while not having a direct quenching effect on complex 9 or the complex 9+G-quadruplex ensemble as shown in FIGS. 5f and 16. These results further validate the method of the present invention can be applied as a screening tool for HCV NS3 helicase inhibitors.

Experimental Section

Materials

Reagents, unless specified, were purchased from Sigma Aldrich (St. Louis, Mo.) and used as received. Iridium chloride hydrate (IrCl₃.xH₂O) was purchased from Precious Metals Online (Australia). Helicase was purchased from Prospec Inc. (Ness-Ziona, Israel). S1 nuclease (S1), endonuclease IV (Endo), DpnI, exonuclease I (ExoI), EcoRI, RNase, DNase, single-stranded DNA binding protein (SSB) was purchased from New England Biolabs Inc. (Beverly, Mass., USA). All oligonucleotides were synthesized by Techdragon Inc. (Hong Kong, China).

General Experimental

Mass spectrometry was performed at the Mass Spectroscopy Unit at the Department of Chemistry, Hong Kong Baptist University, Hong Kong (China). Deuterated solvents for nuclear magnetic resonance (NMR) purposes were obtained from Armar and used as received.

¹H and ¹³C NMR were recorded on a Bruker Avance 400 spectrometer operating at 400 MHz (¹H) and 100 MHz (¹³C). ¹H and ¹³C chemical shifts were referenced internally to solvent shift (acetone-d₆: ¹H d 2.05, ¹³C d 29.8; CD₃Cl: ¹H d 7.26, ¹³C d 76.8). Chemical shifts (8) are quoted in ppm, the downfield direction being defined as positive. Uncertainties in chemical shifts are typically ±0.01 ppm for ¹H and ±0.05 for ¹³C. Coupling constants are typically ±0.1 Hz for ¹H-¹H and ±0.5 Hz for ¹H-¹³C couplings. The following abbreviations are used for convenience in reporting the multiplicity of NMR resonances: s, singlet; d, doublet; t, triplet; q, quartet; m, multiplet; br, broad. All NMR data was acquired and processed using standard Bruker software (Topspin).

Photophysical Measurement

Emission spectra and lifetime measurements for complexes were performed on a PTI TimeMaster C720 Spectrometer (Nitrogen laser: pulse output 337 nm) fitted with a 380 nm filter. Error limits were estimated: λ (±1 nm); τ (±10%); φ (±10%). All solvents used for the lifetime measurements were degassed using three cycles of freeze-vac-thaw.

Luminescence quantum yields were determined using the method of Demas and Crosby [Ru(bpy)₃][PF₆]₂ in degassed acetonitrile as a standard reference solution (Φᵣ=0.062) and calculated according to the following equation:

Φₛ=Φᵣ(Bᵣ/Bₛ)(nₛ/nᵣ)²(Dₛ/Dᵣ)
where the subscripts s and r refer to sample and reference standard solution respectively, n is the refractive index of the solvents, D is the integrated intensity, and Φ is the luminescence quantum yield. The quantity B was calculated by B=1−10^{-AL}, where A is the absorbance at the excitation wavelength and L is the optical path length.

G4-FID Assay

The FID assay was performed as described. The Pu27 G-quadruplex DNA (0.25 μM) in Tris-HCl buffer (20 mM Tris, 100 mM KCl, pH 7.0) were annealed by heating at a temperature of 72-98° C., in particular 95° C., for at least 10 min. Indicated concentration of thiazole orange (0.5 μM for Pu27 G-quadruplex DNA and 0.5 μM for ds17) was added and the mixture was incubated for at least 30 minutes, in particular 1 hour. Emission measurement was taken after addition of each indicated concentration of complex 9 followed by an equilibration time for 5 minutes. The fluorescence area was converted into percentage of displacement (PD) by using the following equation. PD=100−[(FA/FA₀)×100] (FA₀=fluorescence area of DNA-TO complex in the absence of complex 9; FA=fluorescence area in the presence of complex 9).

FRET Melting Assay

The ability of complex 9 to stabilize G-quadruplex DNA was investigated using a fluorescence resonance energy transfer (FRET) melting assay. The labeled G-quadruplex-forming oligonucleotide F21T, consisting of a sequence 5′-FAM-(SEQ ID NO: 5)-TAMRA-3′ (donor fluorophore FAM: 6-carboxyfluorescein; acceptor fluorophore TAMRA: 6-carboxytetramethylrhodamine), was diluted to 200 nM in a potassium cacodylate buffer (100 mM KCl, pH 7.0), and then heated from room temperature to 95° C. in the presence of the indicated concentrations of complex 9. The labeled duplex-forming oligonucleotide F10T, consisting of a sequence 5′-FAM-TATAGCTA-HEG-(SEQ ID NO: 6)-3′ (HEG linker: [(—CH₂—CH₂—O—)₆]), was treated in the same manner, except that the buffer was changed to 10 mM lithium cacodylate (pH 7.4). Fluorescence readings were taken at intervals of 0.5° C. in a range of 25 to 95° C.

Synthesis

In one embodiment of the present invention, luminescent Ir(III) complexes 1 to 17 as shown in FIG. 2 were prepared according to the following method.

A precursor Ir(III) complex dimer [Ir₂(ĈN)₄Cl₂] was prepared. A suspension of [Ir₂(ĈN)₄Cl₂] (0.2 mmol) and a N̂N ligand selected from 1,10-phenanthroline (phen), 2,9-diphenyl-1,10-phenanthroline (2,9-dpphen), 2,9-dimethyl-4,7-diphenyl-1,10-phenanthroline (dmdpphen), 5,6-dimethyl-1,10-phenanthroline (dmphen), 5-chloro-1,10-phenanthroline (chlorophen), 4,7-dichloro-1,10-phenanthroline (dcphen), 2,2′-bipyridine (bpy), 5,5′-dimethyl-2,2′-bipyridine (5,5-dmbpy), 4,7-diphenyl-1,10-phenanthroline (4,7-dpphen), 4,4′-diphenyl-2,2′-bipyridine (dpbpy), and pyrazino[2,3-f][1,10]phenanthroline (pyphen) (0.44 mmol) in a mixture of dichloromethane:methanol (1:1, 20 mL) was refluxed overnight under a nitrogen atmosphere. The resulting solution was then allowed to cool to room temperature, and filtered to remove unreacted cyclometallated dimer. An aqueous solution of ammonium hexafluorophosphate (excess) was added to the filtrate and the filtrate was reduced in volume by rotary evaporation until precipitation of the crude product occurred. The precipitate was then filtered and washed with several portions of water (2×50 mL) followed by diethyl ether (2×50 mL). The product was recrystallized by acetonitrile:diethyl ether vapor diffusion to yield the corresponding complex. The obtained complexes 1 to 17 were characterized by ¹H NMR, ¹³C NMR, high resolution mass spectrometry (HRMS) and elemental analysis.

Complex 1. Yield: 59%. ¹H NMR (400 MHz, Acetone-d₆) δ 8.11-8.09 (d, J=8.0 Hz, 2H), 7.65-7.61 (m, 4H), 7.05-7.01 (d, J=8.0 Hz, 2H), 6.49 (s, 2H), 6.36-6.32 (m, 2H), 6.15-6.03 (m, 10H), 5.86 (s, 2H), 5.69-5.67 (t, J=8.0 Hz, 2H), 5.32-5.30 (t, J=8.0 Hz, 2H), 4.33 (s, 2H); ¹³C NMR (100 MHz, Acetone-d₆) δ 166.9, 150.1, 142.5, 140.6, 140.5, 139.6, 133.0, 131.3, 129.5, 129.3, 128.7, 128.6, 128.5, 128.1, 126.4, 121.9, 112.0, 108.6; HRMS: Calcd for C₄₂H₃₀IrN₆[M−PF₆]⁺: 811.2161 Found: 811.2142; Anal. (C₄₂H₃₀N₆IrPF₆) C, H, N: calcd 52.77, 3.16, 8.79; found 52.54, 3.20, 8.56.

Complex 2. Reported in D.-L. Ma, L.-J. Liu, K.-H. Leung, Y.T. Chen, H.-J. Zhong, D. S.-H. Chan, H.-M. D. Wang and C.-H. Leung, Angew. Chem. Int. Ed, 2014, DOI:10.1002/anie.201404686.

Complex 3. Yield: 53%. ¹H NMR (400 MHz, Acetone-d₆) δ 9.84 (d, J=2.6 Hz, 2H), 9.32 (s, 2H), 8.78 (d, J=8.3 Hz, 2H), 8.26 (d, J=3.7 Hz, 2H), 7.89 (d, J=8.4 Hz, 2H), 7.70 (dd, J=8.0, 1.0 Hz, 2H), 7.09-7.00 (m, 2H), 6.81 (td, J=7.5, 1.2 Hz, 2H), 6.28 (dd, J=7.6, 1.0 Hz, 2H), 2.26 (s, 6H), 1.67 (s, 6H); ¹³C NMR (100 MHz, Acetone-d₆) δ 184.42, 166.53, 154.18, 149.89, 142.75, 140.60, 134.21, 133.43, 130.96, 129.21, 128.29, 127.99, 124.40, 123.26, 113.34, 100.89, 27.44, 11.12; HRMS: Calcd For C₃₆H₃₀IrN₆O₂ [M]⁺: 771.2059 Found: 771.2081; Anal. (C₃₆H₃₀IrN₆O₂PF₆) C, H, N: calcd 47.21, 3.30, 9.18; Found 47.33, 2.92, 9.01.

Complex 4. ¹H NMR (400 MHz, Acetone-d₆) δ 9.84 (d, J=2.4 Hz, 2H), 9.32 (s, 2H), 8.78 (d, J=8.4 Hz, 2H), 8.26 (d, J=3.6 Hz, 2H), 7.89 (d, J=8.4 Hz, 2H), 7.70 (dd, J=8.0, 1.2 Hz, 2H), 7.09-7.00 (m, 2H), 6.80-6.82 (m, 2H), 6.28 (dd, J=7.6, 1.2 Hz, 2H), 2.26 (s, 6H), 1.67 (s, 6H); ¹³C NMR (100 MHz, Acetone-d₆) δ 184.4, 166.5, 154.2, 149.9, 142.8, 140.6, 134.2, 133.4, 131.0, 129.2, 128.3, 128.0, 124.4, 123.3, 113.3, 100.9, 27.4, 11.1; MALDI-TOF-HRMS: Calcd For C₃₆H₃oIrN₆O₂[M−PF₆]⁺: 771.2059 Found: 771.2081; Anal.: (C₃₆H₃₀IrN₆O₂+0.5H₂O) C, H, N: calcd 46.75, 3.38, 9.09; found 46.70, 3.43, 9.07.

Complex 5. Yield: 57%. ¹H NMR (400 MHz, Acetone-d₆) δ 8.58-8.56 (d J=8.0 Hz, 2H), 8.26 (s, 2H), 8.67 (s, 2H), 8.21-8.19 (d, J=8.0 Hz 2H), 8.06-8.04 (d, J=8.0 Hz, 2H), 7.98-7.96 (d, J=8.0 Hz, 2H), 7.83-7.81 (t, J=4.0 Hz, 2H), 7.63-7.60 (m, 10H), 7.16-7.14 (t, J=4.0 Hz, 2H), 7.10-6.98 (d, J=8.0 Hz, 2H), 6.97-6.95 (t, J=4.0Hz, 2H), 6.71-6.99 (d, J=8.0Hz, 2H), 2.07 (s, 6H); ¹³C NMR (100 MHz, Acetone-d₆) δ 169.6, 162.8, 151.4, 150.5, 149.4, 148.7, 146.7, 140.0, 136.6, 134.1, 130.8, 130.7, 130.5, 130.0, 127.8, 127.0, 126.4, 124.8, 123.9, 118.7, 26.3; HRMS: Calcd for C₄₈H₃₆IrN₄ [M−PF₆]⁺: 861.2569, Found: 861.2553; Anal (C₄₈H₃₆N₄IrPF₆+H₂O) C, H, N: calcd 56.30, 3.74, 5.47; found 56.04, 3.42, 5.49.

Complex 6. Reported in F. Gärtner, S. Denurra, S. Losse, A. Neubauer, A. Boddien, A. Gopinathan, A. Spannenberg, H. Junge, S. Lochbrunner, M. Blug, S. Hoch, J. Busse, S. Gladiali and M. Beller, Chem. Eur. J., 2012, 18, 3220-3225.

Complex 7. Reported in K.-H. Leung, H.-Z. He, V. P.Y. Ma, D. S.-H. Chan, C.-H. Leung and D.-L. Ma, Chem. Comm., 2013, 49, 771-773.

Complex 8. Yield: 56%. ¹H NMR (400 MHz, CD₃CN-d₃) δ 8.81-8.79 (d, J=8.0 Hz, 1H), 8.68-8.67 (d, J=4.0 Hz, 1H), 8.61-8.60 (d, J=4.0 Hz, 1H), 8.48-8.46 (d, J=8.0 Hz, 1H), 8.41-8.34 (m, 4H), 8.25-8.22 (d J=8.0 Hz, 2H), 8.15 (s, 1H), 7.98-7.95 (q, J=4.0 Hz, 1H), 7.88-7.85 (q, J=4.0 Hz, 1H), 7.75-7.73 (d, J=8.0 Hz, 2H), 7.28-7.24 (m, 4H), 7.21-7.17 (t, J=8.0 Hz, 2H), 6.92-6.87 (t, J=8.0 Hz, 2H), 6.86-6.81 (t, J=8.0 Hz, 2H), 6.69-6.66 (d, J=8.0 Hz, 2H); ¹³C NMR (100 MHz, CD₃CN-d₃) δ 171.2, 150.7, 150.2, 148.5, 147.1, 147.0, 141.24, 141.21, 139.0, 136.5, 135.7, 135.6, 132.1, 131.7, 131.6, 131.5, 131.0, 130.1, 129.7, 128.6, 128.58, 128.4, 128.39, 128.1, 127.6, 125.1, 125.0, 124.0, 119.0; HRMS: Calcd for C₄₂H₂₇IrN₄Cl [M−PF₆]⁺: 815.1542 Found: 815.1535; Anal (C₄₂H₂₇IrN₄ClPF₆+H₂O) C, H, N: calcd 51.56, 2.99, 5.73; Found 51.75, 2.92, 5.90.

Complex 9. Reported in C. Dragonetti, L. Falciola, P. Mussini, S. Righetto, D. Roberto, R. Ugo, A. Valore, F. De Angelis, S. Fantacci, A. Sgamellotti, M. Ramon and M. Muccini, Inorg. Chem., 2007, 46, 8533-8547.

Complex 10. Yield: 59%. ¹H NMR (400 MHz, Acetone-d₆) δ 8.73 (d J=5.6 Hz, 2H), 8.54 (d, J=8.4 Hz, 2H), 8.48 (d, J=8.4 Hz, 2H), 8.41 (s, 2H), 8.30 (d, J=1.2 Hz, 2H), 8.27 (d, J=8.0 Hz, 2H), 7.81 (d, J=8.0 Hz, 2H), 7.33-7.27 (m, 4H), 7.22 (t, J=8.0 Hz, 2H), 6.97 (t, J=7.6 Hz, 2H), 6.88 (t, J=9.8 Hz, 2H), 6.65 (d J=7.2 Hz, 2H); ¹³C NMR (100 MHz, Acetone-d₆) δ 171.1, 150.9, 150.1, 148.4, 148.3, 147.0, 145.8, 141.3, 135.6, 131.9, 131.5, 130.1, 130.0, 128.8, 128.7, 128.4, 127.7, 126.0, 125.0, 124.1, 119.0; HRMS: Calcd for C₄₂H₂₆Cl₂IrN₄[M−PF₆]⁺: 849.1164, Found: 849.1168. Anal: (C₄₂H₂₆Cl₂IrN₄PF₆+H₂O) C, H, N: calcd 49.81, 2.79, 5.53; found 49.63, 2.85, 5.47.

Complex 11. Yield: 58%. ¹H NMR (400 MHz, Acetone-d₆) δ 8.53 (d, J=8.4 Hz, 2H), 8.47 (d, J=8.8 Hz, 2H), 8.35 (d, J=8.8 Hz, 2H), 8.06 (d, J=8.0 Hz, 2H), 7.88 (d, J=8.0 Hz, 2H), 7.82-7.79 (m, 4H), 7.44 (d, J=8.8 Hz, 2H), 7.37 (t, J=1.2 Hz, 2H), 7.08 (t, J=8.0 Hz, 2H), 6.99 (t, J=8.0 Hz, 2H), 6.81 (t, J=1.2 Hz, 2H), 6.49 (d, J=8.0 Hz, 2H), 2.81 (s, 6H); ¹³C NMR (100 MHz, Acetone-d₆) δ 171.8, 165.4, 149.2, 148.9, 148.6, 147.1, 141.0, 139.5, 134.0, 131.5, 131.1, 130.1, 130.0, 128.6, 128.4, 128.0, 127.4, 127.3, 124.8, 123.5, 118.2, 25.2; HRMS: calcd for C₄₄H₃₂IrN₄[M−PF₆]⁺: 809.2256 found: 809.2304. Anal: (C₄₄H₃₂IrN₄PF₆+2H₂O) C, H, N: calcd 53.38, 3.67, 5.66; found 53.10, 3.50, 5.65.

Complex 12. Yield: 63%. ¹HNMR (400 MHz; Acetone-d₆): δ 8.52 (d, J=8.5 Hz, 2H), 8.34 (d, J=8.9 Hz, 2H), 8.06 (dd, J=7.9, 1.2 Hz, 2H), 7.96 (dd, J=8.1, 1.4 Hz, 2H), 7.77 (s, 2H), 7.68-7.62 (m, 10H), 7.51 (dd, J=7.4, 2.1 Hz, 4H), 7.46 (ddd, J=8.0, 7.0, 1.0 Hz, 2H), 7.15-7.06 (m, 4H), 6.85-6.81 (m, 2H), 6.58 (dd, J=7.8, 0.9 Hz, 2H), 2.08 (s, 6H). ¹³C NMR (100 MHz; Acetone-d₆): δ 170.9, 163.9, 150.5, 148.75, 148.55, 147.6, 146.1, 140.0, 135.8, 133.4, 130.6, 130.1, 129.60, 129.53, 129.19, 129.10, 127.8, 127.35, 127.22, 126.8, 126.6, 124.2, 124.0, 122.6, 117.4, 24.3. MALDI-TOF-HRMS: Calcd: 961.2880, Found: 961.2846. Anal. Calcd for C₅₆H₄₀F₆IrN₄P+2H₂O, C, 58.89; H, 3.88, N, 4.91, Found: C, 59.115; H, 3.58; N, 4.935.

Complex 13. ¹H NMR (400 MHz, Acetone-d₆) δ 9.62 (d, J=8.0 Hz, 2H), 9.19 (s, 2H), 8.87 (d, J=5.2 Hz, 2H), 8.57 (d, J=8.4 Hz, 2H), 8.49 (d, J=8.4 Hz, 2H), 8.34 (d, J=1.2 Hz, 2H), 8.32-8.23 (m, 2H), 7.79 (d, J=8.2 Hz, 2H), 7.38 (d, J=8.4 Hz, 2H), 7.27-7.23 (m, 4H), 6.92-6.71 (m, 4H), 6.69 (d, J=0.8 Hz, 2H); ¹³C NMR (100 MHz, Acetone-d₆) δ 171.3, 151.3, 151.1, 149.0, 148.5, 147.8, 147.1, 141.3, 140.0, 136.1, 135.6, 131.7, 131.5, 130.6, 130.1, 128.9, 128.7, 128.4, 127.6, 125.3, 124.1, 119.0; HRMS: Calcd for C₄₄H₂₈IrN₆[M−PF₆]⁺: 833.2005, Found: 833.1926. Anal: (C₄₄H₂₈IrN₆PF₆+2.5H₂O) C, H, N: calcd51.66, 3.25, 8.32; found 51.77, 3.08, 8.64.

Complex 14. Yield: 60%. ¹H NMR (400 MHz, Acetone-d₆) δ 8.94 (d, J=1.6 Hz, 2H), 8.55-8.53 (m, 4H), 8.87 (d, J=5.2 Hz, 2H), 8.39 (d, J=6.0 Hz, 2H), 8.28 (d, J=7.6 Hz, 2H), 8.02 (d, J=5.6 Hz, 2H), 7.90-7.88 (m, 6H), 7.58-7.53 (m, 8H), 7.43 (t, J=8.0 Hz, 2H), 7.20-7.18 (m, 4H), 6.86 (t, J=8.0 Hz, 2H), 6.61 (d, J=8.0 Hz, 2H); ¹³C NMR (100 MHz, Acetone-d₆) δ 171.3, 157.2, 152.3, 151.8, 149.1, 148.5, 147.0, 141.3, 136.2, 135.4, 132.0, 131.6, 131.5, 130.3, 128.9, 128.4, 127.7, 126.4, 125.8, 123.8, 122.7, 119.0; HRMS: Calcd for Cs₂H₃₆IrN₆[M−PF₆]⁺: 909.2569 Found: 909.2590. Anal: (C5₂H₃₆IrN₆PF₆) C, H, N: calcd59.25, 3.44, 5.32; found 59.03, 3.63, 5.10.

Complex 15. Yield: 54%. ¹H NMR (400 MHz, Acetone-d₆) δ 9.09 (d, J=8.4 Hz, 1H), 8.89 (d, J=7.6 Hz, 1H), 8.81 (d, J=4.8 Hz, 1H), 8.72 (d, J=4.8 Hz, 1H), 8.61 (s, 1H), 8.27-8.23 (m, 1H), 8.16-8.13 (m, 1H), 7.93 (d, J=7.2 Hz, 2H), 7.55 (d, J=8.0 Hz, 2H), 7.16 (t, J=8.0 Hz, 2H), 7.09 (t, J=7.6 Hz, 2H), 6.93-6.90 (m, 2H), 6.79-6.75 (m, 2H), 6.54-6.51 (m, 2H), 5.54 (t, J=7.6 Hz, 2H); ¹³C NMR (100 MHz, Acetone-d₆) δ 164.7, 153.1, 152.5, 149.5, 149.2, 149.0, 147.5, 139.7, 139.6, 137.9, 135.4, 134.3, 133.4, 133.3, 133.2, 131.5, 130.7, 130.6, 130.3, 129.1, 127.5, 127.4, 124.2, 124.1, 123.7, 123.6, 123.5, 122.5, 113.4, 113.3, 112.8; HRMS: Calcd for C₃₈H₂₅ClIrN₆[M−PF₆]⁺: 793.1458 Found: 793.1422. Anal.: (C₃₈H₂₅ClIrN₆PF₆+H₂O) C, H, N: calcd47.73, 2.85, 8.79; found 47.75, 3.16, 8.50.

Complex 16. Yield: 53%. ¹H NMR (400 MHz, Acetone-d₆) δ 9.04 (dd, J=8.5, 1.3 Hz, 1H), 8.84 (dd, J=8.3, 1.3 Hz, 1H), 8.62 (s, 1H), 8.54 (dd, J=5.1, 1.3 Hz, 1H), 8.46 (dd, J=5.0, 1.4 Hz, 1H), 8.18 (dd, J=8.5, 5.1 Hz, 1H), 8.07 (dd, J=8.3, 5.1 Hz, 1H), 7.45-7.41 (m, 2H), 7.29 (dd, J=7.3, 2.5 Hz, 2H), 7.20-7.13 (m, 2H), 7.06 (tt, J=7.4, 1.2 Hz, 2H), 5.00 (dtd, J=10.6, 8.6, 2.3 Hz, 2H), 4.60 (dtd, J=10.6, 8.6, 5.2 Hz, 2H), 3.76 (dddd, J=12.0, 10.7, 8.5, 3.4 Hz, 2H), 3.08 (dddd, J=11.9, 10.8, 7.9, 3.9 Hz, 2H); ¹³C NMR (100 MHz, Acetone-d₆) δ 181.34, 153.95, 153.43, 150.56, 150.30, 149.60, 148.13, 138.31, 135.79, 133.87, 133.31, 132.18, 131.66, 131.19, 130.05, 128.21, 128.08, 127.63, 122.88, 72.39, 50.31; MALDI-TOF-HRMS: Calcd For C₃₀H₂₃ClIrN₄O₂ [M]⁺: 699.1126, Found: 699.1136; Anal: (C₃₀H₂₃ClIrN₄O₂PF₆) C, H, N: calcd42.68, 2.75, 6.64, found 42.98, 2.87, 6.71.

Complex 17. Reported in P. M. Griffiths, F. Loiseau, F. Puntoriero, S. Serroni and S. Campagna, Chem.Comm., 2000, 2297-2298.

Total Cell Extract Preparation

The TRAMPC1 (ATCC® CRL2730™) cell line were purchased from American Type Culture Collection (Manassas, Va. 20108 USA). Prostate cancer cells were trypsinized and resuspended in TE buffer (10 mM Tris-HCl 7.4, 1 mM EDTA). After incubation on ice for 10 minutes, the lysate was centrifuged and the supernatant was collected.

Luminescence Response of Ir(III) Complexes 1-17 Towards Different Forms of DNA

The G-quadruplex DNA-forming sequence (PS2. M) was annealed in Tris-HCl buffer (20 mM Tris, 100 mM KCl, pH 7.0) and were stored at −20° C. or less than −20° C. before use. Complex 1-17 (1 μM) was added to 5 μM of ssDNA, dsDNA or PS2. M G-quadruplex DNA in Tris-HCl buffer (20 mM Tris, pH 7.0).

Detection of Enzymes Activities

The random-coil oligonucleotides ON1 (100 μM) and ON2 (100 μM) were incubated in Tris buffer (20 mM, pH 7.0). The solution was heated at a temperature of 72-98° C., in particular 95° C., for at least 30 seconds in particular 10 min, cooled to room temperature at 0.1° C./s, and further incubated at room temperature for 1 hour to ensure formation of the duplex substrate. The annealed product was stored at −20° C. before use. For assaying enzyme activity, 50 μL of Tris buffered solution (5 mM Tris-HCl, 5 mM NaCl, 1 mM MgCl₂, 1 mM ATP, 0.1 mM DTT, pH 7.9) with the indicated concentrations of helicase or S1, Endo, DpnI, ExoI, EcoRI, RNase, DNase, and SSB were added to a solution containing the duplex substrate (0.25 μM). The mixture was heated to 37° C. for at least 30 minutes, e.g. 2 hours, to allow the indicated enzymes-catalyzed unwinding of the duplex substrate to take place. The duplex unwinding reaction was quenched by the addition of EDTA at a final concentration of 20 mM, and the mixture was subsequently diluted using Tris buffer (20 mM Tris, 20 mM KCl, 150 mM NH₄Ac, pH 7.2) to a final volume of 500 μL. Finally, 1 μM of complex 9 or suramin, TBBT and ciprofloxacin were added to the mixture. Emission spectra were recorded in the 500-720 nm range using an excitation wavelength of 360 nm.

For the detection of helicase activity in cell extract, 50 μL of Tris buffered solution (5 mM Tris-HCl, 5 mM NaCl, 1 mM MgCl₂, 1 mM ATP, 0.1 mM DTT, pH 7.9) and the indicated concentrations of helicase were added to a solution containing the duplex substrate (0.25 μM) and cell extract. The mixture was heated to 37° C. for at least 30 minutes, e.g. 2 hours, to allow the helicase-catalyzed unwinding of the duplex substrate to take place. The duplex unwinding reaction was quenched by the addition of EDTA at a final concentration of 20 mM, and the mixture was subsequently diluted using Tris buffer (20 mM Tris, 20 mM KCl, 150 mM NH₄Ac, pH 7.2) to a final volume of 500 μL. Finally, 1 μM of complex 9 was added to the mixture. Emission spectra were recorded in the 500-720 nm range using an excitation wavelength of 360 nm.

TABLE 1
DNA sequences used in the present invention:

| | Sequence |
|---|---|
| PS2.M | SEQ ID NO: 1 |
| ON1 | SEQ ID NO: 2 |
| ON2 | SEQ ID NO: 3 |
| ON1ₘ | SEQ ID NO: 4 |
| F21T | 5′-FAM-(SEQ ID NO: 5)-TAMRA-3′ |
| F10T | 5′-FAM-TATAGCTA-HEG-(SEQ ID NO: 6)-3′ |
| ds17 | SEQ ID NO: 7 |
| | SEQ ID NO: 8 |
| CCR5-DEL | SEQ ID NO: 9 |

TABLE 2
Photophysical properties of Ir(III) complexes 1-17.

| Com- | Quantum | λₑₘ/ | Lifetime/ | UV/vis absorption |
|---|---|---|---|---|
| plex | yield | nm | μs | λ_{abs}/nm (ε/dm³ mol⁻¹ cm⁻¹) |
| 1 | 0.13 | 629 | 3.29 | 269 (4.19 × 10⁴), 354 (1.27 × 10⁴), |
| | | | | 370 (1.54 × 10⁵) |
| 2 | 0.057 | 577 | 0.74 | 261 (3.3 × 10⁴), 268 (3.2 × 10³), |
| | | | | 296 (1.9 × 10⁴), 371 (9.05 × 10³) |
| 3 | 0.089 | 567 | 4.16 | 261 (1.24 × 10⁴), 311 (5.48 × 10³), |
| | | | | 348 (1.73 × 10³) |
| 4 | 0.015 | 578 | 1.53 | 231 (3.49 × 10⁴), 270 (2.56 × 10⁴), |
| | | | | 339 (5.32 × 10³) |
| 5 | 0.12 | 590 | 1.23 | 336 (1.42 × 10⁴) |
| 6 | 0.089 | 580 | 0.28 | 236 (1.56 × 10⁵), 285 (7.75 × 10⁴), |
| | | | | 302 (6.43 × 10⁴) |
| 7 | 0.056 | 571 | 1.34 | 278 (1.33 × 10⁴), 332 (4.67 × 10³) |
| 8 | 0.27 | 583 | 4.31 | 280 (3.6 × 10⁴), 429 (5.9 × 10³) |
| 9 | 0.12 | 570 | 8.13 | 270 (5.72 × 10⁴), 333 (2.06 × 10⁴), |
| 10 | 0.086 | 590 | 2.89 | 263 (2.90 × 10⁴), 278 (2.99 × 10⁴), |
| | | | | 332 (1.10 × 10⁴) |
| 11 | 0.087 | 570 | 1.96 | 270 (3.13 × 10⁴), 337 (2.33 × 10⁴) |
| 12 | 0.063 | 575 | 1.84 | 234 (2.55 × 10⁴), 262 (2.20 × 10⁴), |
| | | | | 286 (2.67 × 10⁴), 350 (7.91 × 10³) |
| 13 | 0.067 | 568 | 4.61 | 262 (3.79 × 10⁴), 279 (2.88 × 10⁴), |
| | | | | 334 (1.13 × 10⁴) |
| 14 | 0.15 | 560 | 4.586 | 278 (1.34 × 10⁵), 355 (1.92 × 10⁴), |
| | | | | 454 (4.0 × 10³) |
| 15 | 0.092 | 620 | 2.71 | 274 (7.38 × 10³), 301 (6.31 × 10³), |
| | | | | 372 (1.93 × 10³) |
| 16 | 0.069 | 588 | 1.09 | 230 (2.73 × 10⁴), |
| | | | | 270 (1.64 × 10⁴), 345 (3.33 × 10³) |
| 17 | 0.078 | 608 | 2.87 | 235 (1.69 × 10⁴), 252 (1.81 × 10⁴), |
| | | | | 266 (1.94 × 10⁴) |

A library of 17 luminescent Ir(III) complexes containing various ĈN and N̂N ligands were screened according to the present invention for their ability to act as G-quadruplex probes. In the preferred embodiment, Ir(III) complex 9 was used to be a G-quadruplex-selective luminescent probe. The inventors also developed a label-free luminescent assay for helicase activity utilizing the G-quadruplex-selective property of complex 9. Compared to previously reported radiographic or luminescent assays that require multiple steps and/or the use of isotopically or fluorescently labeled nucleic acids, the present invention's label-free approach is faster and cost-effective as expensive and tedious pre-labeling or immobilization steps are avoided. On the other hand, the labeling of an oligonucleotide with a fluorophore may disrupt the interaction between the oligonucleotide with its cognate target. Finally, the present invention developed a label-free DNA-based detection platform employs luminescent transition metal complexes, which offer several advantages compared to the relatively more popular organic fluorophores, such as long phosphorescence lifetimes, large Stokes shift values and modular syntheses. Additionally, the assay could function effectively in diluted cell extract, and its application for the screening of helicase inhibitors was also demonstrated. It is envisioned that the present invention can be applied in various applications, in particular in biochemical and biomedical research.

If desired, the different functions discussed herein may be performed in a different order and/or concurrently with each other. Furthermore, if desired, one or more of the above-described features may be optional or may be combined. Those skilled in the art will appreciate that the invention described herein is susceptible to variations and modifications other than those specifically described.

Other aspects and advantages of the invention will be apparent to those skilled in the art from a review of the preceding description.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other technical terms used herein have the same meaning as commonly understood to one of ordinary skill in the art to which the invention belongs.

Citation or identification of any reference in this document shall not be construed as an admission that such reference is available as prior art for the present application.

1. A method of detecting helicase activity in a test solution, comprising the steps of:
(a) providing a substrate solution containing a double-stranded DNA, wherein said double-stranded DNA is a substrate for a helicase to be detected;
(b) introducing the substrate solution to the test solution to form a reaction solution;
(c) applying a luminescent probe into the reaction solution to form a mixture; and
(d) measuring a luminescent response of the luminescent probe in the mixture, wherein the luminescent response corresponds to the helicase activity in the test solution.

2. The method according to claim 1, wherein in step (b), the double-stranded DNA is unwound to provide a single-stranded DNA and a G-quadruplex-forming DNA in the presence of the helicase, in which the G-quadruplex-forming DNA can fold to form a G-quadruplex DNA.

3. The method according to claim 2, wherein the luminescent probe interacts with the G-quadruplex DNA to generate the luminescent response.

4. The method according to claim 2, wherein the G-quadruplex DNA comprises a sequence of SEQ ID NO: 1.

5. The method according to claim 1, wherein the luminescent probe is a luminescent iridium (III) complex, the luminescent iridium (III) complex comprises:
an iridium-containing dimer; and
a nitrogen-containing ligand having at least two nitrogen atoms, wherein the nitrogen-containing ligand is a derivative of pyridine.

6. The method according to claim 5, wherein the iridium-containing dimer ligand has at least two benzene rings; and the nitrogen-containing ligand is selected from the group consisting of 2,9-diphenyl-1,10-phenanthroline, 2,9-dimethyl-4,7-diphenyl-1,10-phenanthroline, 5,6-dimethyl-1,10-phenanthroline, 5-chloro-1,10-phenanthroline, 4,7-dichloro-1,10-phenanthroline, 5,5′-dimethyl-2,2′-bipyridine, 4,7-diphenyl-1,10-phenanthroline, 4,4′-diphenyl-2,2′-bipyridine, pyrazino[2,3-f][1,10]phenanthroline and a derivative thereof.

7. The method according to claim 1, wherein the test solution comprises cells.

8. The method according to claim 1, wherein the helicase is hepatitis C virus helicase.

9. A luminescent iridium (III) complex comprising,
an iridium-containing dimer; and
a nitrogen-containing ligand having at least two nitrogen atoms, wherein the nitrogen-containing ligand is a derivative of pyridine.

10. The luminescent iridium (III) complex according to claim 9, wherein the nitrogen-containing ligand is selected from the group consisting of 2,9-diphenyl-1,10-phenanthroline, 2,9-dimethyl-4,7-diphenyl-1,10-phenanthroline, 5, 6-dimethyl-1,10-phenanthroline, 5-chloro-1,10-phenanthroline, 4, 7-dichloro-1,10-phenanthroline, 5,5′-dimethyl-2,2′-bipyridine, 4,7-diphenyl-1,10-phenanthroline, 4,4′-diphenyl-2,2′-bipyridine, pyrazino[2,3-f][1,10]phenanthroline and a derivative thereof.

11. The luminescent iridium (III) complex according to claim 9, wherein the iridium-containing dimer comprises at least two benzene rings.

12. The luminescent iridium (III) complex according to claim 11, wherein the iridium-containing dimer comprises a chemical structure selected from the group consisting of the following structures:

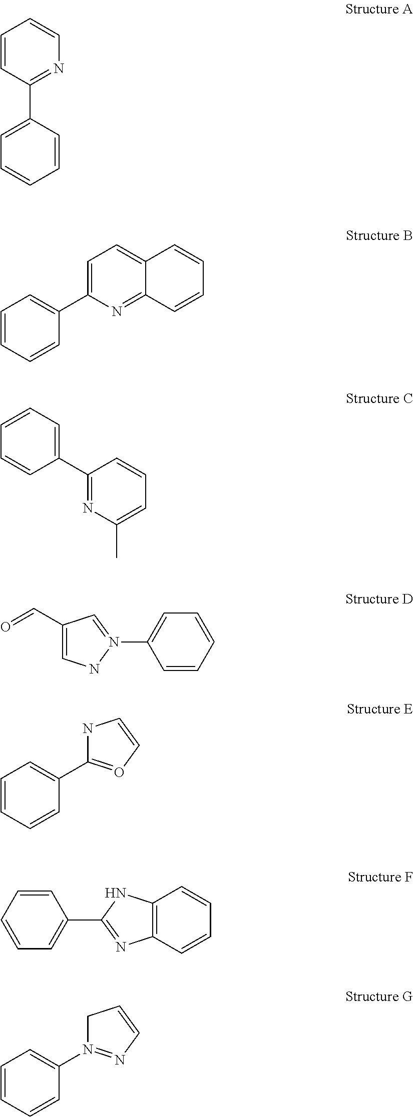

13. The luminescent iridium (III) complex according to claim 12, wherein the iridium (III) complex comprises one of the following structures:

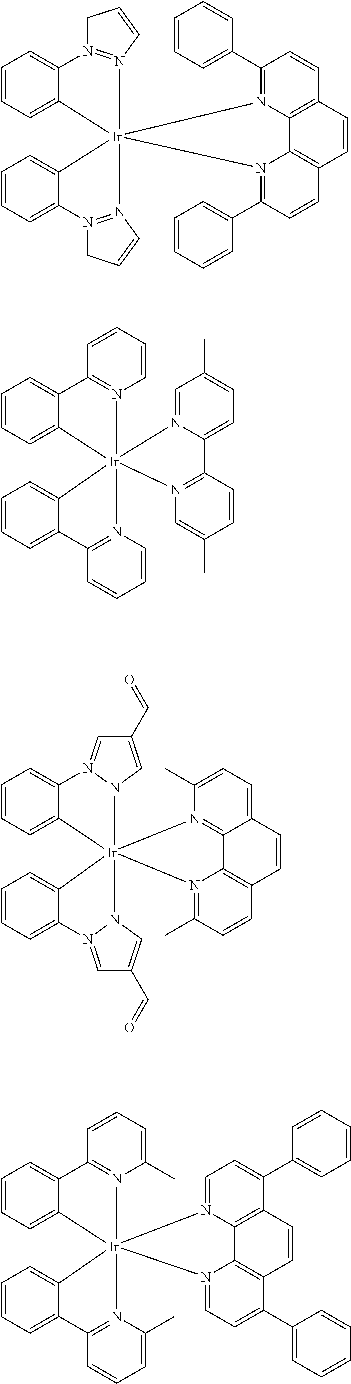

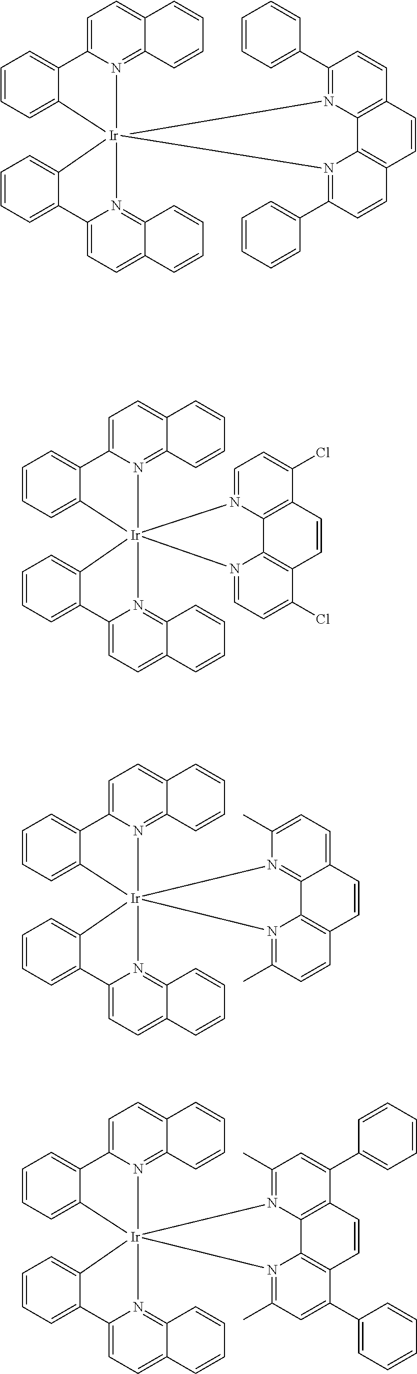

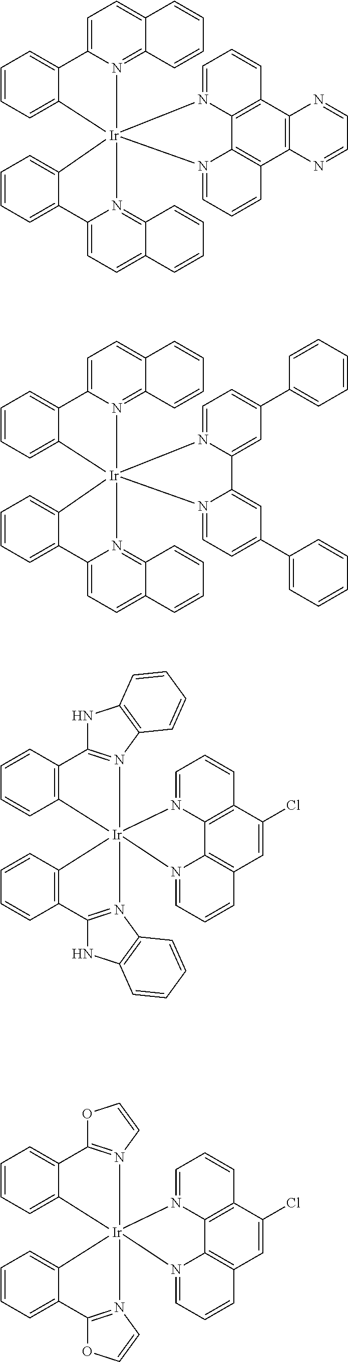

14. A method of screening a luminescent probe for detecting helicase activity, comprising the steps of:
(i) measuring luminescent responses of a luminescent probe candidate towards a G-quadruplex DNA, a double-stranded DNA, and a single-stranded DNA respectively; and
(ii) determining selectivity of the luminescent probe candidate towards the G-quadruplex DNA over the double-stranded DNA, and the single-stranded DNA based on the luminescent responses measured in step (i).

15. The method according to claim 14, wherein the helicase is capable of unwinding the double-stranded DNA to provide the single-stranded DNA and a G-quadruplex-forming DNA in which the G-quadruplex-forming DNA folds to form the G-quadruplex DNA.

16. The method according to claim 14, wherein the G-quadruplex DNA comprises a sequence of SEQ ID NO: 1.

17. The method according to claim 14, wherein the selectivity of the luminescent probe candidate towards the G-quadruplex DNA over the double-stranded DNA is determined if a ratio between the luminescent response of the luminescent probe candidate towards the G-quadruplex DNA and that towards the double-stranded DNA is larger than 1; and the selectivity of the luminescent probe candidate towards the G-quadruplex DNA over the single-stranded DNA is determined if a ratio between the luminescent response of the luminescent probe candidate towards the G-quadruplex DNA and that towards the single-stranded DNA is larger than 1.

18. The method according to claim 14, wherein the helicase is a hepatitis C virus NS3 helicase.

19. The method according to claim 14, further comprising the step of:
(iii) measuring a luminescent response of the luminescent probe candidate towards the helicase.

20. The method according to claim 19, wherein the selectivity of the luminescent probe candidate towards the G-quadruplex DNA over the helicase is determined if a ratio between the luminescent response of the luminescent probe candidate towards the G-quadruplex DNA and that towards the helicase is larger than 1.


SEQUENCE LISTING (9 sequences)

SEQ ID NO: 1 - DNA, 18 bp, Artificial Sequence
  A designed G-quadruplex DNA-forming sequence
gtgggtagggcgggttgg

SEQ ID NO: 2 - DNA, 54 bp, Artificial Sequence
  A designed G-quadruplex-forming sequence
gtgggtagggcgggttggtggcgacggcagcgaggcagaggagcagagggagca

SEQ ID NO: 3 - DNA, 29 bp, Artificial Sequence
  A complementary sequence of SEQ ID No. 2
gcctcgctgccgtcgccaccaacccgccc

SEQ ID NO: 4 - DNA, 54 bp, Artificial Sequence
  A designed mutant DNA sequence
gtatatataccgggttggtggcgacggcagcgaggcagaggagcagagggagca

SEQ ID NO: 5 - DNA, 21 bp, Artificial Sequence
  A G-quadruplex-forming oligonucleotide
gggttagggttagggttaggg

SEQ ID NO: 6 - DNA, 11 bp, Artificial Sequence
  A duplex-forming oligonucleotide
tatagctatat

SEQ ID NO: 7 - DNA, 17 bp, Artificial Sequence
  A DNA strand
ccagttcgtagtaaccc

SEQ ID NO: 8 - DNA, 17 bp, Artificial Sequence
  A complementary strand of SEQ ID No. 7
gggttactacgaactgg

SEQ ID NO: 9 - DNA, 29 bp, Artificial Sequence
  A DNA strand
ctcattttccatacattaaagatagtcat